(19) United States
(12) Patent Application Publication
(10) Pub. No.: US 2004/0077656 A1
(43) Pub. Date: Apr. 22, 2004

(54) Quinolines and nitrogenated derivatives thereof substituted in 4-position by a piperazine-containing moiety and their use as antibacterial agents

(76) Inventors: Roger Edward Markwell, Harlow (GB); Neil David Pearson, Harlow (GB); Christian Smethurst, Harlow (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/450,892

(22) PCT Filed: Dec. 19, 2001
(86) PCT No.: PCT/GB01/05661

(30) Foreign Application Priority Data
Dec. 20, 2000 (GB) 0031086.2

(51) Int. Cl. A61K 31/496; C07D 413/14; C 07D 4 3/02

(52) U.S. Cl. 514/253.05; 514/253.06; 544/363

(57) ABSTRACT

Piperazine derivatives, containing a quinoline analog moiety, of formula (I) and pharmaceutically acceptable derivatives thereof useful in methods of treatment of bacterial infections in mammal, particularly in man.

This invention relates to novel compounds, compositions containing them and their use as antibacterials.

WO99/37635, WO00/21948 and WO00/21952 disclose piperidine derivatives having antibacterial activity.

EP0579263, EP0742207, JP2169569 and EP0296560, generically disclose piperazine compounds as acetylcholinesterase inhibitors and sigma receptor antagonists

WO92/17475, WO98/02438, WO97/03069 and WO96/39145 disclose certain bicyclic heteroaromatic compounds having cholinesterase inhibitor, protein tyrosine kinase inhibitor, cell proliferation inhibitor and human epidermal growth factor receptor type 2 inhibitor activity.

JP7179407 discloses bicyclic heteroaromatic compounds having GPIIb/IIIa inhibitory activity

WO97/17973 discloses piperazine derivatives having hemoregulatory activities.

WO99/05096 discloses naphthamidine compounds having urokinase inhibitory activity.

This invention provides a compound of formula (I) or a pharmaceutically acceptable derivative thereof:

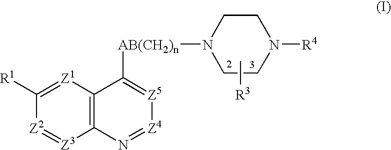

wherein:

one of Z¹, Z², Z³, Z⁴ and Z⁵ is N, one is CR^{1a} and the remainder are CH, or one of Z¹, Z², Z³, Z⁴ and Z⁵ is CR^{1a} and the remainder are CH;

R¹ and R^{1a} are independently selected from hydrogen; hydroxy; (C₁₋₆) alkoxy optionally substituted by (C₁₋₆)alkoxy, amino, piperidyl, guanidino or amidino any of which is optionally N-substituted by one or two (C₁₋₆)alkyl, acyl or (C₁₋₆)alkylsulphonyl groups, (C₁₋₆)alkylthio, heterocyclylthio, heterocyclyloxy, arylthio, aryloxy, acylthio, acyloxy or (C₁₋₆)alkylsulphonyloxy; (C₁₋₆)alkoxy-substituted (C₁₋₆)alkyl; halogen; (C₁₋₆)alkyl; (C₁₋₆)alkylthio; trifluromethyl; nitro; azido; acyl; acyloxy; acylthio; (C₁₋₆)alkylsulphonyl; (C₁₋₆)alkylsulphoxide; arylsulphonyl; arylsulphoxide or an amino, piperidyl, guanidino or amidino group optionally N-substituted by one or two (C₁₋₆)alkyl, acyl or (C₁₋₆)alylsulphonyl groups,

provided that when none of Z¹, Z², Z³, Z⁴ and Z⁵ is N, then R¹ is not hydrogen;

R³ is hydrogen; or

R³ is in the 2- or 3-position and is:

carboxy; (C₁₋₆)alkoxycarbonyl; aminocarbonyl wherein the amino group is optionally substituted by hydroxy, (C₁₋₆)alkyl, hydroxy(C₁₋₆)alkyl, aminocarbonyl(C₁₋₆)alkyl, (C₂₋₆)alkenyl, (C₁₋₆)alkylsulphonyl, trifluoromethylsulphonyl, (C₂₋₆)alkenylsulphonyl, (C₁₋₆)alkoxycarbonyl, (C₁₋₆)alkylcarbonyl, (C₂₋₆)alkenyloxycarbonyl or (C₂₋₆)alkenylcarbonyl and optionally further substituted by (C₁₋₆)alkyl, hydroxy(C₁₋₆)alkyl, aminocarbonyl(C₁₋₆)alkyl or (C₂₋₆)alkenyl; cyano; tetrazolyl; 2-oxo-oxazolidinyl optionally substituted by R¹⁰; 3-hydroxy-3-cyclobutene-1,2-dione-4-yl; 2,4-thiazolidinedione-5-yl; tetrazol-5-ylaminocarbonyl; 1,2,4-triazol-5-yl optionally substituted by R¹⁰; or 5-oxo-1,2,4-oxadiazol-3-yl; or

(C₁₋₄)alkyl or ethenyl optionally substituted with any of the groups listed above for R³ and/or 0 to 2 groups R¹² independently selected from:

halogen; (C₁₋₆)alkylthio; trifluoromethyl; (C₁₋₆)alkoxycarbonyl; (C₁₋₆)alkylcarbonyl; (C₂₋₆)alkenyloxycarbonyl; (C₂₋₆)alkenylcarbonyl; hydroxy optionally substituted by (C₁₆)alkyl, (C₂₋₆)alkenyl, (C₁₋₆)alkoxycarbonyl, (C₁₋₆)alkylcarbonyl, (C₂₋₆)alkenyloxycarbonyl, (C₂₋₆)alkenylcarbonyl or aminocarbonyl wherein the amino group is optionally substituted by (C₁₋₆)alkyl, (C₂₋₆)alkenyl, (C₁₋₆)alkylcarbonyl or (C₂₋₆)alkenylcarbonyl; amino optionally mono- or disubstituted by (C₁₋₆)alkoxycarbonyl, (C₁₋₆)alkylcarbonyl, (C₂₋₆)alkenyloxycarbonyl, (C₂₋₆)alkenylcarbonyl, (C₁₋₆)allyl, (C₂₋₆)alkenyl, (C₁₋₆)alkylsulphonyl, (C₂₋₆)alkenylsulphonyl or aminocarbonyl wherein the amino group is optionally substituted by (C₁₋₆)alkyl or (C₂₋₆)alkenyl; aminocarbonyl wherein the amino group is optionally substituted by (C₁₋₆)alkyl, hydroxy(C₁₋₆)alkyl, aminocarbonyl(C₁₋₆)alkyl, (C₂₋₆)alkenyl, (C₁₋₆)alkoxycarbonyl, (C₁₋₆)alkylcarbonyl, (C₂₋₆)alkenyloxycarbonyl or (C₂₋₆)alkenylcarbonyl and optionally further substituted by (C₁₋₆)alkyl, hydroxy(C₁₋₆)alkyl, aminocarbonyl(C₁₋₆)alkyl or (C₂₋₆)alkenyl; oxo; (C₁₋₆)alkylsulphonyl; (C₂₋₆)alkenylsulphonyl; or (C₁₋₆)aminosulphonyl wherein the amino group is optionally substituted by (C₁₋₆)alkyl or (C₂₋₆)alkenyl;

in addition when R³ is disubstituted with a hydroxy or amino containing substituent and a carboxy containing substituent these may optionally together form a cyclic ester or amide linkage, respectively;

R¹⁰ is selected from (C₁₋₄)alkyl; (C₂₋₄)alkenyl and aryl any of which may be optionally substituted by a group R¹² as defined above; carboxy; aminocarbonyl wherein the amino group is optionally substituted by hydroxy, (C₁₋₆)alkyl, (C₂₋₆)alkenyl, (C₁₋₆)alkylsulphonyl, trifluoromethylsulphonyl, (C₂₋₆)alkenylsulphonyl, (C₁₋₆)alkoxycarbonyl, (C₁₋₆)alkylcarbonyl, (C₂₋₆)alkenyloxycarbonyl or (C₂₋₆)alkenylcarbonyl; and

R⁴ is a group —V—X¹—X²—X³—X⁴ in which:

V is CH₂, CO or SO₂;

X¹ is CR¹⁴R¹⁵;

X² is NR¹³, O, SO₂ or CR¹⁴R¹⁵;

X³ is NR¹³, O or CR¹⁴R¹⁵; wherein:

each of R¹⁴ and R¹⁵ is independently selected from: hydrogen; (C₁₋₄)alkoxy; (C₁₋₄)alkylthio; trifluoromethyl; cyano; (C₁₋₄)alkyl; (C₂₋₄)alkenyl; (C₁₋₄)alkoxycarbonyl; (C₁₋₄)alkylcarbonyl; (C₂₋₄)alkenyloxycarbonyl; (C₂₋₄)alkenylcarbonyl; hydroxy, amino or aminocarbonyl optionally substituted as for corresponding substituents in R³; (C₁₋₄)alkylsulphonyl; (C₂₋₄)alkenylsulphonyl; or aminosulphonyl wherein the amino group is optionally substituted by (C₁₋₄)alkyl or (C₂₋₄)alkenyl, provided that R¹⁴ and R¹⁵ on the same carbon atom are not both selected from optionally substituted hydroxy and optionally substituted amino; or

R¹⁴ and R¹⁵ together represent oxo;

R¹³ is hydrogen; trifluoromethyl, (C₁₋₆)alkyl; (C₂₋₆)alkenyl; (C₁₋₆)alkoxycarbonyl; (C₁₋₆)alkylcarbonyl; aminocarbonyl wherein the amino group is optionally substituted by (C₁₋₆)alkoxycarbonyl, (C₁₋₆)alkylcarbonyl, (C₂₋₆)alkenyloxycarbonyl, (C₂₋₆)alkenylcarbonyl, (C₁₋₆)allyl or (C₂₋₆)alkenyl and optionally further substituted by (C₁₋₆)alkyl or (C₂₋₆)alkenyl;

two R¹⁴ groups or an R¹³ and an R¹⁴ group in X¹, X² and X³ together with the atoms to which they are attached and, if appropriate, the intervening group X² form a 5 or 6 membered carbocyclic or heterocyclic ring and the remaining R¹³, R¹⁴ and R¹⁵ groups are as above defined; or

two R¹⁴ groups or an R¹³ and an R¹⁴ group on adjacent atoms together represent a bond and the remaining R¹³, R¹⁴ and R¹⁵ groups are as above defined;

X⁴ is phenyl or C or N linked monocyclic aromatic 5- or 6-membered heterocycle containing up to four heteroatoms selected from O, S and N and optionally C-substituted by up to three groups selected from (C₁₋₄)alkylthio; halo; carboxy(C₁₋₄)alkyl; halo(C₁₋₄)alkoxy; halo(C₁₋₄)alkyl; (C₁₋₄)alkyl; (C₂₋₄)alkenyl; (C₁₋₄)alkoxycarbonyl; formyl; (C₁₋₄)alkylcarbonyl; (C₂₋₄)alkenyloxycarbonyl; (C₂₋₄)alkenylcarbonyl; (C₁₋₄)alkylcarbonyloxy; (C₁₋₄)alkoxycarbonyl(C₁₋₄)alkyl; hydroxy; hydroxy(C₁₋₄)alkyl; mercapto(C₁₋₄)alkyl; (C₁₋₄)alkoxy; nitro; cyano; carboxy; amino or aminocarbonyl optionally substituted as for corresponding substituents in R³; (C₁₋₄)alkylsulphonyl; (C₂₋₄)alkenylsulphonyl; or aminosulphonyl wherein the amino group is optionally substituted by (C₁₋₄)alkyl or (C₂₋₄)alkenyl; aryl, aryl(C₁₋₄)alkyl or aryl(C₁₋₄)alkoxy; and

optionally N substituted by trifluoromethyl; (C₁₋₄)alkyl optionally substituted by hydroxy, (C₁₋₆)alkoxy, (C₁₋₆)alkylthio, halo or trifluoromethyl; (C₂₋₄)alkenyl; aryl; aryl(C₁₋₄)alkyl; (C₁₋₄)alkoxycarbonyl; (C₁₋₄)alkylcarbonyl; formyl; (C₁₋₆)alkylsulphonyl; or aminocarbonyl wherein the amino group is optionally substituted by (C₁₋₄)alkoxycarbonyl, (C₁₋₄)alkylcarbonyl, (C₂₋₄)alkenyloxycarbonyl, (C₂₋₄)alkenylcarbonyl, (C₁₋₄)alkyl or (C₂₋₄)alkenyl and optionally further substituted by (C₁₋₄)alkyl or (C₂₋₄)alkenyl;

n is 0 and AB is NR¹¹CO, CO—CR⁸R⁹, CR⁶R⁷—CO, NR¹¹SO₂, CR⁶R⁷—SO₂ or CR⁶R⁷—CR⁸R⁹, provided that R⁸ and R⁹ are not optionally substituted hydroxy or amino and R⁶ and R⁸ do not represent a bond:

or n is 1 and AB is NR¹¹CO, CO—CR⁸R⁹, CR⁶R⁷—CO, NR¹¹SO₂, CONR¹¹, CR⁶R⁷—CR⁸R⁹, O—CR⁸R⁹ or NR¹¹—CR⁸R⁹;

and wherein:

each of R⁶ and R⁷, R⁸ and R⁹ is independently selected from: H; (C₁₋₆)alkoxy; (C₁₋₆)alkylthio; halo; trifluoromethyl; azido; (C₁₋₆)alkyl; (C₂₋₆)alkenyl; (C₁₋₆)alkoxycarbonyl; (C₁₋₆)alkylcarbonyl; (C₂₋₆)alkenyloxycarbonyl; (C₂₋₆)alkenylcarbonyl; hydroxy, amino or aminocarbonyl optionally substituted as for corresponding substituents in R³; (C₁₋₆)alkylsulphonyl; (C₂₋₆)alkenylsulphonyl; or (C₁₋₆)aminosulphonyl wherein the amino group is optionally substituted by (C₁₋₆)alkyl or (C₂₋₆)alkenyl;

or R⁶ and R⁸ together represent a bond and R⁷ and R⁹ are as above defined;

and each R¹¹ is independently H, trifluoromethyl, (C₁₋₆)alkyl, (C₂₋₆)alkenyl, (C₁₋₆)alkoxycarbonyl, (C₁₋₆)alkylcarbonyl, aminocarbonyl wherein the amino group is optionally substituted by (C₁₋₆)alkoxycarbonyl, (C₁₋₆)alkylcarbonyl, (C₂₋₆)alkenyloxycarbonyl, (C₂₋₆)alkenylcarbonyl, (C₁₋₆)alkyl or (C₂₋₆)alkenyl and optionally further substituted by (C₁₋₆)alkyl or (C₂₋₆)alkenyl;

or where one of R³ and R⁶, R⁷, R⁸ or R⁹ contains a carboxy group and the other contains a hydroxy or amino group they may together form a cyclic ester or amide linkage;

and each R¹¹ is independently H; trifluromethyl; (C₁₋₆)alkyl; (C₂₋₆)alkenyl; (C₁₋₆)alkoxycarbonyl; (C₁₋₆)alkylcarbonyl; or aminocarbonyl wherein the amino group is optionally substituted by (C₁₋₆)alkoxycarbonyl, (C₁₋₆)alkylcarbonyl, (C₂₋₆)alkenyloxycarbonyl, (C₂₋₆)alkenylcarbonyl, (C₁₋₆)alkyl or (C₂₋₆)alkenyl and optionally further substituted by (C₁₋₆)alkyl or (C₂₋₆)alkenyl;

or where one of R³ and R⁶, R⁷, R⁸ or R⁹ contains a carboxy group and the other contains a hydroxy or amino group they may together form a cyclic ester or amide linkage.

The invention also provides the use of a compound of formula (I) or a pharmaceutically acceptable derivative thereof in the manufacture of a medicament for use in the treatment of bacterial infections in mammals.

The invention also provides a pharmaceutical composition for use in the treatment of bacterial infections in mammals comprising a compound of formula (I), or a pharmaceutically acceptable derivative thereof, and a pharmaceutically acceptable carrier.

The invention further provides a method of treatment of bacterial infections in mammals, particularly in man, which method comprises the administration to a mammal in need of such treatment of an effective amount of a a compound of formula (I), or a pharmaceutically acceptable derivative thereof.

In one aspect, when X⁴ is optionally substituted phenyl, —V—X¹—X²—X³— is not —(CH₃)₃O—, —CH₂—(C₃-alkenyl)- or CH₃)₃CO—.

In another aspect, when Z5 is N, AB(CH₂)ₙ is (CH₂)₂, (CH₂)₃, CH═CHCH₂, NR¹¹(CH₂)₂ or O(CH₂)₂ and —V—X¹—X²—X³— is (CH₂)₄, X4 is not optionally substituted phenyl or an optionally substituted monocyclic aromatic 5-membered heterocycle.

Preferably Z⁵ is CH or N, Z³ is CH or CF and Z¹, Z² and Z⁴ are each CH, or Z¹ is N, Z³ is CH or CF and Z², Z⁴ and Z⁵ are each CH.

When R¹ or R^{1a} is substituted alkoxy it is preferably (C₂₋₆)alkoxy substitituted by optionally N-substituted amino, guanidino or amidino, or (C₁₋₆)alkoxy substituted by piperidyl. Suitable examples of R¹ alkoxy include methoxy, n-propyloxy, i-butyloxy, aminoethyloxy, aminopropyloxy, aminobutyloxy, aminopentyloxy, guanidinopropyloxy, piperidin-4-ylmethyloxy, phthalimido pentyloxy or 2-aminocarbonylprop-2-oxy.

Preferably R¹ is methoxy, amino(C₃₋₅)alkyloxy, guanidino(C₃₋₅)alkyloxy, piperidyl(C₃₋₅)alkyloxy, nitro or fluoro; more preferably methoxy, amino(C₃₋₅)alkyloxy or guanidino(C₃₋₅)alkyloxy. Preferably R^{1a} is H or F. Most preferably R¹ is methoxy and R^{1a} is H or when Z³ is CR^{1a} it may be C—F.

When Z⁵ is CR^{1a}, R^{1a} is preferably hydrogen, cyano, hydroxymethyl or carboxy, most preferably hydrogen.

Preferably n is 0.

Preferred examples of R³ include hydrogen; (C₁₋₄) alkyl; ethenyl; optionally substituted 1-hydroxy-(C₁₋₄) alkyl; optionally substituted aminocarbonyl; carboxy(C₁₋₄)alkyl; optionally substituted aminocarbonyl(C₁₋₄)alkyl; cyano(C₁₋₄)alkyl; optionally substituted 2-oxo-oxazolidinyl and optionally substituted 2-oxo-oxazolidinyl(C₁₋₄alkyl). More preferred R³ groups are hydrogen; CONH₂; 1-hydroxyalkyl e.g. CH₂OH, CH(OH)CH₂CN; CH₂CO₂H; CH₂CONH₂; —CONHCH₂CONH₂; 1,2-dihydroxyalkyl e.g. CH(OH)CH₂OH; CH₂CN; 2-oxo-oxazolidin-5-yl and 2-oxo-oxazolidin-5-yl(C₁₋₄alkyl). Most preferably R³ is hydrogen.

When R³ and R⁶, R⁷, R⁸ or R⁹ together form a cyclic ester or amide linkage, it is preferred that the resulting ring is 5-7 membered. It is further preferred that the group A or B which does not form the ester or amide linkage is CH₂.

When A is CH(OH) the R-stereochemistry is preferred.

Preferably A is NH, NCH₃, CH₂, CHOH, CH(NH₂), C(Me)(OH) or CH(Me).

Preferably B is CH₂ or CO.

Preferably n=0.

Most preferably:

n is 0 and either A is CHOH and B is CH₂ or A is NH and B is CO.

Preferably R¹¹ is hydrogen or (C₁₋₄)alkyl e.g. methyl, more preferably hydrogen.

V is preferably CH₂.

R¹³, R¹⁴ and R¹⁵ are preferably H or (C₁₋₄)alkyl such as methyl, more preferably hydrogen.

X¹ is preferably CH₂.

X² is preferably CH₂ or together with X³ forms a CH═CH group.

X³ is preferably CH₂, O or NH, or together with X² forms a CH═CH group.

Preferred linker groups —X¹—X²—X³— include —(CH₂)₂—O—, —CH₂—CH═CH—, —(CH₂)₃—, —(CH₂)₂—NH—, —CH(OH)—CH₂—NH—, —CH₂NHCO— or —CH₂CONH—. Where the linker group is a cyclised groups, two R¹⁴ groups or R¹³ and R¹⁴ complete a carbocylic or heterocyclic ring such as oxazolidin-2-one. The cyclised linker group —X¹—X²—X³— is preferably oxazolidin-2-one-3,5-diyl where X³ is N.

Monocyclic aromatic heterocyclic groups for X⁴ include pyridyl, pyrazinyl, pyrimidinyl, triazolyl, tetrazolyl, thienyl, isoimidazolyl, thiazolyl, furanyl and imidazolyl, all preferably C-linked, and N-linked 2H-pyridazone and 1H-pyrid-2-one. Preferred aromatic heterocyclic groups include pyrid-2-yl, pyrid-3-yl, 1,3-thiazole-2-yl, pyrimidin-2-yl, pyrimidin-5-yl and fur-2-yl.

Heterocyclic X⁴ is preferably unsubstituted or substituted by a group selected from halo especially fluoro, trifluoromethyl and nitro, most preferably substituted by fluoro.

Preferred substituents on phenyl X⁴ include halo, especially fluoro, nitro, cyano, trifluoromethyl, methyl, methoxycarbonyl and methylcarbonylamino.

Preferably X⁴ is 2-pyridyl, 3-fluorophenyl, 3,5-difluorophenyl or 1,3-thiazole-2-yl.

When used herein, the term “alkyl” includes groups having straight and branched chains, for instance, methyl, ethyl, n-propyl, iso-propyl, n-butyl, iso-butyl, sec-butyl, tbutyl, pentyl and hexyl. The term ‘alkenyl’ should be interpreted accordingly.

Halo or halogen includes fluoro, chloro, bromo and iodo.

Haloalkyl moieties include 1-3 halogen atoms.

Unless otherwise defined, the term ‘heterocyclic’ as used herein includes aromatic and non-aromatic, single and fused, rings suitably containing up to four hetero-atoms in each ring selected from oxygen, nitrogen and sulphur, which rings may be unsubstituted or C-substituted by, for example, up to three groups selected from (C₁₋₄)alkylthio; halo; carboxy(C₁₋₄)alkyl; halo(C₁₋₄)alkoxy; halo(C₁₋₄)alkyl; (C₁₋₄)alkyl; (C₂₋₄)alkenyl; (C₁₋₄)alkoxycarbonyl; formyl; (C₁₋₄)alkylcarbonyl; (C₂₋₄)alkenyloxycarbonyl; (C₂₋₄)alkenylcarbonyl; (C₁₋₄)alkylcarbonyloxy; (C₁₋₄)alkoxycarbonyl(C₁₋₄)alkyl; hydroxy; hydroxy(C₁₋₄)alkyl; mercapto(C₁₋₄)alkyl; (C₁₋₄)alkoxy; nitro; cyano; carboxy; amino or aminocarbonyl optionally substituted as for corresponding substituents in R³; (C₁₋₄)alkylsulphonyl; (C₂₋₄)alkenylsulphonyl; or aminosulphonyl wherein the amino group is optionally substituted by (C₁₋₄)alkyl or (C₂₋₄)alkenyl; optionally substituted aryl, aryl(C₁₋₄)alkyl or aryl(C₁₋₄)alkoxy and oxo groups. Each heterocyclic ring suitably has from 4 to 7, preferably 5 or 6, ring atoms. A fused heterocyclic ring system may include carbocyclic rings and need include only one heterocyclic ring. Compounds within the invention containing a heterocyclyl group may occur in two or more tautometric forms depending on the nature of the heterocyclyl group; all such tautomeric forms are included within the scope of the invention.

Where an amino group forms part of a single or fused non-aromatic heterocyclic ring as defined above suitable optional substituents in such substituted amino groups include H; trifluoromethyl; (C₁₋₄)alkyl optionally substituted by hydroxy, (C₁₋₆)alkoxy, (C₁₋₆)alkylthio, halo or trifluoromethyl; (C₂₋₄)alkenyl; aryl; aryl (C₁₋₄)alkyl; (C₁₋₄)alkoxycarbonyl; (C₁₋₄)alkylcarbonyl; formyl; (C₁₋₆)alkylsulphonyl; or aminocarbonyl wherein the amino group is optionally substituted by (C₁₋₄)alkoxycarbonyl, (C₁₋₄)alkylcarbonyl, (C₂₋₄)alkenyloxycarbonyl, (C₂₋₄)alkenylcarbonyl, (C₁₋₄)alkyl or (C₂₋₄)alkenyl and optionally further substituted by (C₁₋₄)alkyl or (C₂₋₄)alkenyl.

When used herein the term ‘aryl’, includes phenyl and naphthyl, each optionally substituted with up to five, preferably up to three, groups selected from(C₁₋₄)alkylthio; halo; carboxy(C₁₋₄)alkyl; halo(C₁₋₄)alkoxy; halo(C₁₋₄)alkyl; (C₁₋₄)alkyl; (C₂₋₄)alkenyl; (C₁₋₄)alkoxycarbonyl; formyl; (C₁₋₄)alkylcarbonyl; (C₂₋₄)alkenyloxycarbonyl; (C₂₋₄)alkenylcarbonyl; (C₁₋₄)alkylcarbonyloxy; (C₁₋₄)alkoxycarbonyl(C₁₋₄)alkyl; hydroxy; hydroxy(C₁₋₄)alkyl; mercapto(C₁₋₄)alkyl; (C₁₋₄)alkoxy; nitro; cyano, carboxy; amino or aminocarbonyl optionally substituted as for corresponding substituents in R³; (C₁₋₄)alkylsulphonyl; (C₂₋₄)alkenylsulphonyl; or aminosulphonyl wherein the amino group is optionally substituted by (C₁₋₄)alkyl or (C₂₋₄)alkenyl; phenyl, phenyl(C₁₋₄)allyl or phenyl(C₁₋₄)alkoxy.

The term ‘acyl’ includes (C₁₋₆)alkoxycarbonyl, formyl or (C₁₋₆) alkylcarbonyl groups.

Preferred compounds of formula (I) include:

3 3-(3,5-Difluoro-phenyl)-5-{4-[(R)-2-hydroxy-2-(6-methoxy-quinolin-4-yl)-ethyl]-piperazin-1-ylmethyl}-oxazolidin-2-one

3,5-Difluoro-N-(2-{4-[(R)-2-hydroxy-2-(6-methoxy-quinolin-4-yl)-ethyl]-piperazin-1-yl}-ethyl)-benzamide

(R)-1-(6-Methoxy-quinolin-4-yl)-2-[4-(4-phenyl-butyl)-piperazin-1-yl]-ethanol

N-(3,5-Difluoro-phenyl)-3-{4-[(R)-2-hydroxy-2-(6-methoxy-quinolin-4-yl)-ethyl]-piperazin-1-yl}-propionamide

3-(3-Fluoro-phenyl)-5-{4-[(R)-2-hydroxy-2-(6-methoxy-quinolin-4-yl)-ethyl]-piperazin-1-ylmethyl}-oxazolidin-2-one

and pharmaceutically acceptable derivatives thereof.

Some of the compounds of this invention may be crystallised or recrystallised from solvents such as organic solvents. In such cases solvates may be formed. This invention includes within its scope stoichiometric solvates including hydrates as well as compounds containing variable amounts of water that may be produced by processes such as lyophilisation.

Since the compounds of formula (I) are intended for use in pharmaceutical compositions it will readily be understood that they are each provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and preferably from 10 to 59% of a compound of the formula (I) or pharmaceutically acceptable derivative thereof.

Pharmaceutically acceptable derivatives of the above-mentioned compounds of formula (I) include the free base form or their acid addition or quaternary ammonium salts, for example their salts with mineral acids e.g. hydrochloric, hydrobromic, sulphuric nitric or phosphoric acids, or organic acids, e.g. acetic, fumaric, succinic, maleic, citric, benzoic, p-toluenesulphonic, methanesulphonic, naphthalenesulphonic acid or tartaric acids. Compounds of formula (I) may also be prepared as the N-oxide. Compounds of formula (I) having a free carboxy group may also be prepared as an in vivo hydrolysable ester. The invention extends to all such derivatives.

Examples of suitable pharmaceutically acceptable in vivo hydrolysable ester-forming groups include those forming esters which break down readily in the human body to leave the parent acid or its salt. Suitable groups of this type include those of part formulae (i), (ii), (iii), (iv) and (v):

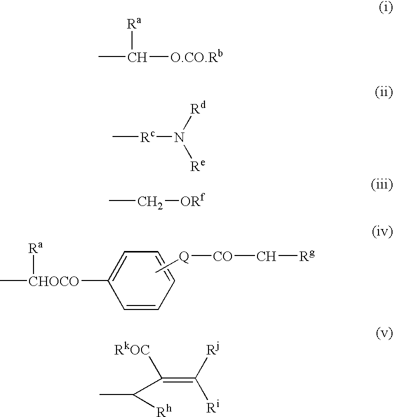

wherein R^{a} is hydrogen, (C₁₋₆) alkyl, (C₃₋₇) cycloalkyl, methyl, or phenyl, R^{b} is (C₁₋₆) alkyl, (C₁₋₆) alkoxy, phenyl, benzyl, (C₃₋₇) cycloalkyl, (C₃₋₇) cycloalkyloxy, (C₁₋₆) alkyl (C₃₋₇) cycloalkyl, 1-amino (C₁₆) alkyl, or 1-(C₁₋₆ alkyl)amino (C₁₋₆) alkyl; or R^{a} and R^{b} together form a 1,2-phenylene group optionally substituted by one or two methoxy groups; R^{c} represents (C₁₋₆) alkylene optionally substituted with a methyl or ethyl group and R^{d} and R^{e} independently represent (C₁₋₆) alkyl; R^{f} represents (C₁₋₆) alkyl; R^{g} represents hydrogen or phenyl optionally substituted by up to three groups selected from halogen, (C₁₋₆) alkyl, or (C₁₋₆) alkoxy; Q is oxygen or NH; R^{h} is hydrogen or (C₁₋₆) alkyl; Rⁱ is hydrogen, (C₁₋₆) alkyl optionally substituted by halogen, (C₂₋₆) alkenyl, (C₁₋₆) alkoxycarbonyl, aryl or heteroaryl; or R^{h} and Rⁱ together form (C ₁₋₆) alkylene; R^{j} represents hydrogen, (C₁₋₆) alkyl or (C₁₋₆) alkoxycarbonyl; and R^{k} represents (C₁₈) alkyl, (C₁₈) alkoxy, (C₁₆) alkoxy(C₁₋₆)alkoxy or aryl.

Examples of suitable in vivo hydrolysable ester groups include, for example, acyloxy(C₁₋₆)alkyl groups such as acetoxymethyl, pivaloyloxymethyl, α-acetoxyethyl, α-pivaloyloxyethyl, 1-(cyclohexylcarbonyloxy)prop-1-yl, and (1-aminoethyl)carbonyloxymethyl; (C₆)alkoxycarbonyloxy(C₁₋₆)alkyl groups, such as ethoxycarbonyloxymethyl, α-ethoxycarbonyloxyethyl and propoxycarbonyloxyethyl; di(C₁₋₆)alkylamino(C₁₋₆)alkyl especially di(C₁₋₄)alkylamino(C₁₋₄)alkyl groups such as dimethylaminomethyl, dimethylaminoethyl, diethylaminomethyl or diethylaminoethyl; 2-((C₁₋₆)alkoxycarbonyl)-2-(C₂₋₆)alkenyl groups such as 2-(isobutoxycarbonyl)pent-2-enyl and 2-(ethoxycarbonyl)but-2-enyl; lactone groups such as phthalidyl and dimethoxyphthalidyl.

A further suitable pharmaceutically acceptable in vivo hydrolysable ester-forming group is that of the formula:

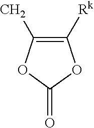

wherein R^{k} is hydrogen, C₁₋₆ alkyl or phenyl.

R is preferably hydrogen.

Certain of the above-mentioned compounds of formula (I) may exist in the form of optical isomers, e.g. diastereoisomers and mixtures of isomers in all ratios, e.g. racemic mixtures. The invention includes all such forms, in particular the pure isomeric forms. For examples the invention includes compound in which an A-B group CH(OH)—CH₂ is in either isomeric configuration the R-isomer is preferred. The different isomeric forms may be separated or resolved one from the other by conventional methods, or any given isomer may be obtained by conventional synthetic methods or by stereospecific or asymmetric syntheses.

In a further aspect of the invention there is provided a process for preparing compounds of formula (I), or a pharmaceutically acceptable derivative thereof, which process comprises reacting a compound of formula (IV) with a compound of formula (V):

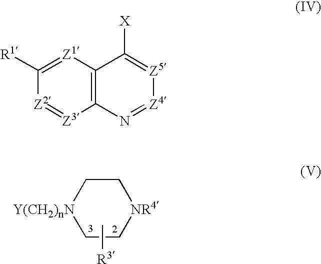

wherein n is as defined in formula (I); Z^{1′}, Z^{2′}, Z^{3′}, Z^{4′}, Z^{5′}, R^{1′}, R^{3′} and R^{4′} are Z¹, Z², Z³, Z⁴, Z⁵, R¹, R³ and R⁴ as defined in formula (I) or groups convertible thereto;

and X and Y may be the following combinations:

(i) X is A′-COW, Y is H and n is 0;

(ii) X is CR⁶═CR⁸R⁹, Y is H and n is 0;

(iii) X is oxirane, Y is H and n is 0;

(iv) X is N═C═O and Y is H and n is 0;

(v) one of X and Y is CO₂R^{y} and the other is CH₂CO₂R^{x};

(vi) X is CHR⁶R⁷ and Y is C(═O)R⁸;

(vii) X is CR⁷═PR^{z}₃ and Y is C(═O)R⁹ and n=1;

(viii) X is C(═O)R⁷ and Y is CR⁹═PR^{z}₃ and n=1;

(ix) Y is COW and X is NHR^{11′}, NCO or NR^{11′}COW and n=0 or 1 or when n=1 X is COW and Y is NHR^{11′}, NCO or NR^{11′}COW;

(x) X is NHR^{11′} and Y is C(═O)R⁸ and n=1;

(xi) X is NHR^{11′} and Y is CR⁸R⁹W and n=1;

(xii) X is NR^{11′}COCH₂W or NR^{11′}SO₂CH₂W and Y is H and n=0;

(xiii) X is CR⁶R⁷SO₂W and Y is H and n=0;

(xiv) X is W or OH and Y is CH₂OH and n is 1;

(xv) X is NHR^{11′} and Y is SO₂W or X is NR^{11′}SO₂W and Y is H, and n is 0;

in which W is a leaving group, e.g. halo or imidazolyl; R^{x} and R^{y} are (C₁₋₆)alkyl; R^{z} is aryl or (C₁₋₆)alkyl; A′ and NR^{11′} are A and NR¹¹ as defined in formula (I), or groups convertible thereto; and oxirane is:

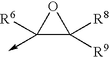

wherein R⁶, R⁸ and R⁹ are as defined in formula (I);

and thereafter optionally or as necessary converting A′, Z^{1′}, Z^{2′}, Z^{3′}, Z^{4′}, Z^{5′}, R^{1′}, R^{3′}, R^{4′} and NR^{11′}; to A, Z¹, Z², Z³, Z⁴, Z⁵, R¹, R³, R⁴ and NR^{11′}; converting A-B to other A-B, interconverting R¹, R³ and/or R⁴, and/or forming a pharmaceutically acceptable derivative thereof.

Process variant (i) initially produces compounds of formula (I) wherein A-B is A′-CO.

Process variant (ii) initially produces compounds of formula (I) wherein A-B is CHR⁶—CR⁸R⁹.

Process variant (iii) initially produces compounds of formula (I) wherein A-B is CR⁶(OH)—CR⁸R⁹.

Process variant (iv) initially produces compounds of formula (I) where A-B is NH—CO.

Process variant (v) initially produces compounds of formula (I) wherein A-B is CO—CH₂ or CH₂—CO.

Process variant (vi) initially produces compounds of formula (I) wherein A-B is CR⁶R⁷—CR⁸OH.

Process variant (vii) and (viii) initially produce compounds of formula (I) wherein A-B is CR⁶═CR⁸.

Process variant (ix) initially produces compounds of formula (I) where A-B is CO—NR¹¹ or NR¹—CO.

Process variant (x) initially produces compounds of formula (I) wherein A-B is NR¹¹—CHR⁸.

Process variant (xi) initially produces compounds of formula (I) wherein A-B is NR^{11′}—CR⁸R⁹.

Process variant (xii) initially produces compounds of formula (I) where A-B is NR^{11′}—CO or NR^{11′}SO₂ and n=1.

Process variant (xiii) initially produces compounds of formula (I) where A-B is CR⁶R⁷—SO₂.

Process variant (xiv) initially produces compounds of formula (I) wherein A-B is O—CH₂.

Process variant (xv) initially produces compounds where AB is NR¹¹SO₂.

In process variants (i) and (ix) the reaction is a standard amide or urea formation reaction involving e.g.:

1. Activation of a carboxylic acid (e.g. to an acid chloride, mixed anhydride, active ester, O-acyl-isourea or other species), and treatment with an amine (Ogliaruso, M. A.; Wolfe, J. F. in The Chemistry of Functional Groups (Ed. Patai, S.) Suppl. B: The Chemistry of Acid Derivatives, Pt. 1 (John Wiley and Sons, 1979), pp 442-8; Beckwith, A. L. J. in The Chemistry of Functional Groups (Ed. Patai, S.) Suppl. B: The Chemistry of Amides (Ed. Zabricky, J.) (John Wiley and Sons, 1970), p 73 ff. The acid and amine are preferably reacted in the presence of an activating agent such as 1-(dimethylaminopropyl)-3-ethylcarbodiimide hydrochloride (EDC) or 1-hydroxybenzotriazole (HOBT); or

2. The specific methods of:

a. in situ conversion of an acid into the amine component by a modified Curtius reaction procedure (Shioiri, T., Murata, M., Hamada, Y., Chem. Pharm. Bull. 1987, 35, 2698)

b. in situ conversion of the acid component into the acid chloride under neutral conditions (Villeneuve, G. B.; Chan, T. H., Tetrahedron. Lett. 1997, 8, 6489).

A′ may be, for example, protected hydroxymethylene.

The process variant (ii) is a standard addition reaction using methods well known to those skilled in the art. The process is preferably carried out in a polar organic solvent e.g. acetonitrile in the presence of an organic base e.g. triethylamine.

In process variant (iii) the coupling may be effected in acetonitrile at room temperature in the presence of one equivalent of lithium perchlorate as catalyst (general method of J. E. Chateauneuf et al, J. Org. Chem., 56, 5939-5942, 1991) or more preferably with ytterbium triflate in dichloromethane. In some cases an elevated temperature such as 40-70° C. may be beneficial. Alternatively, the piperazine may be treated with a base, such as one equivalent of butyl lithium, and the resulting salt reacted with the oxirane in an inert solvent such as tetrahydrofuran, preferably at an elevated temperature such as 80° C. Use of a chiral epoxide will afford single diastereomers. Alternatively, mixtures of diastereomers may be separated by preparative HPLC or by conventional resolution through crystallisation of salts formed from chiral acids.

The process variant (iv) is a standard urea formation reaction from the reaction of an isocyanate with an amine and is conducted by methods well known to those skilled in the art (for example see March, J, Advanced Organic Chemistry, Edition 3 (John Wiley and Sons, 1985), p802-3). The process is preferably carried out in a polar solvent such as N,N-dimethylformamide.

In process variant (v) the process is two step: firstly a condensation using a base, preferably sodium hydride or alkoxide, sodamide, alkyl lithium or lithium dialkylamide, preferably in an aprotic solvent e.g. ether, THF or benzene; secondly, hydrolysis using an inorganic acid, preferably HCl in aqueous organic solvent at 0-100° C. Analogous routes are described in DE330945, EP31753, EP53964 and H. Sargent, J. Am. Chem. Soc. 68, 2688-2692 (1946). Similar Claisen methodology is described in Soszko et. al., Pr. Kom. Mat. Przyr. Poznan. Tow. Przyj. Nauk., (1962), 10, 15.

In process variant (vi) the reaction is carried out in the presence of a base, preferably organometallic or metal hydride e.g. NaH, lithium diisopropylamide or NaOEt, preferably in an aprotic solvent, preferably THF, ether or benzene at −78 to 25° C. (analogous process in Gutswiller et al. (1978) J. Am. Chem. Soc. 100, 576).

In process variants (vii) and (viii) if a base is used it is preferably NaH, KH, an alkyl lithium e.g. BuLi, a metal alkoxide e.g. NaOEt, sodamide or lithium dialkylamide e.g. di-isopropylamide. An analogous method is described in U.S. Pat. No. 3,989,691 and M. Gates et. al. (1970) J. Amer. Chem. Soc., 92, 205, as well as Taylor et al. (1972) JACS 94, 6218.

In process variant (x) where Y is CHO the reaction is a standard reductive alkylation using, e.g., sodium borohydride or sodium triacetoxyborohydride (Gribble, G. W. in Encyclopedia of Reagents for Organic Synthesis (Ed. Paquette, L. A.) (John Wiley and Sons, 1995), p 4649).

The process variant (xi) is a standard alkylation reaction well known to those skilled in the art, for example where an alcohol or amine is treated with an alkyl halide in the presence of a base (for example see March, J; Advanced Organic Chemistry, Edition 3 (John Wiley and Sons, 1985), p364-366 and p342-343). The process is preferably carried out in a polar solvent such as N,N-dimethylformamide

In process variant (xii) the reaction is an alkylation, examples of which are described in J. Med. Chem. (1979) 22(10) 1171-6. The compound of formula (IV) may be prepared from the corresponding compound where X is NHR^{11′} by acylation with an appropriate derivative of the acid WCH₂COOH such as the acid chloride or sulphonation with an appropriate derivative of the sulphonic acid WCH₂SO₃H such as the sulphonyl chloride.

In process variant (xiii) the reaction is a standard sulphonamide formation reaction well known to those skilled in the art. This may be e.g. the reaction of a sulphonyl halide with an amine.

In process variant (xiv) where X is W such as halogen, methanesulphonyloxy or trifluoromethanesulphonyloxy, the hydroxy group in Y is preferably converted to an OM group where M is an alkali metal by treatment of an alcohol with a base. The base is preferably inorganic such as NaH, lithium diisopropylamide or sodium. Where X is OH, the hydroxy group in Y is activated under Mitsunobu conditions (Fletcher et. al. J Chem Soc. (1995), 623). Alternatively the X═O and Y═CH₂OH groups can be reacted directly by activation with dichlorocarbodiimide (DCC) (Chem. Berichte 1962, 95, 2997 or Angewante Chemie 1963 75, 377).

In process variant (xv) the reaction is conducted in the presence of an organic base such as triethylamine or pyridine such as described by Fuhrman et. al., J. Amer. Chem. Soc.; 67, 1245, 1945. The X═NR¹¹′SO₂W or Y═SO₂W intermediates can be formed from the requisite amine e.g. by reaction with SO₂Cl₂ analogously to the procedure described by the same authors Fuhrman et. al., J. Amer. Chem. Soc.; 67, 1245, 1945.

Reduction of a carbonyl group A or B to CHOH can be readily accomplished using reducing agents well known to those skilled in the art, e.g. sodium borohydride in aqueous ethanol or lithium aluminium hydride in ethereal solution. This is analogous to methods described in EP53964, U.S. Pat. No. 384,556 and J. Gutzwiller et al, J. Amer. Chem. Soc., 1978, 100, 576.

The carbonyl group A or B may be reduced to CH₂ by treatment with a reducing agent such as hydrazine in ethylene glycol, at e.g. 130-160° C., in the presence of potassium hydroxide.

Reaction of a carbonyl group A or B with an organometallic reagent yields a group where R⁶ or R⁸ is OH and R⁷ or R⁹ is alkyl.

A hydroxy group on A or B may be oxidised to a carbonyl group by oxidants well known to those skilled in the art, for example, manganese dioxide, pyridinium chlorochromate or pyridinium dichromate.

A hydroxyalkyl A-B group CHR⁶CR⁸OH or CR⁶(OH)CHR⁸ may be dehydrated to give the group CR CR⁸ by treatment with an acid anhydride such as acetic anhydride.

Methods for conversion of CR⁶═CR⁸ by reduction to CHR⁶CHR⁸ are well known to those skilled in the art, for example using hydrogenation over palladium on carbon as catalyst. Methods for conversion of CR⁶═CR⁸ to give the A-B group CR⁶(OH)CHR⁸ or CHR⁶CR⁸OH are well known to those skilled in the art for example by epoxidation and subsequent reduction by metal hydrides, hydration, hydroboration or oxymercuration.

An amide carbonyl group may be reduced to the corresponding amine using a reducing agent such as lithium aluminium hydride.

A hydroxy group in A or B may be converted to azido by activation and displacement e.g. under Mitsunobu conditions using hydrazoic acid or by treatment with diphenylphosphorylazide and base, and the azido group in turn may be reduced to amino by hydrogenation.

Examples of groups Z^{1′}, Z^{2′}, Z^{3′}, Z^{4′}, Z^{5′}, are CR^{1a′} where R^{1a′} is a group convertible to R^{1a}, Z^{1′}, Z^{2′}, Z^{3′}, Z^{4′} and Z^{5′} are preferably Z¹, Z², Z³, Z⁴ and Z⁵.

R^{1a′} and R¹ are preferably R^{1a} and R¹. R^{1′} is preferably methoxy. R^{3′} is R³ or more preferably hydrogen, vinyl, alkoxycarbonyl or carboxy. R^{4′} is R⁴ or more preferably H or an N-protecting group such as t-butoxycarbonyl, benzyloxycarbonyl or 9-fluorenylmethyloxycarbonyl.

Conversions of R^{1a′}, R^{1′}, R^{3′} and R^{4′} and interconversions of R^{1a}, R¹, R³ and R⁴ are conventional. In compounds which contain an optionally substituted hydroxy group, suitable conventional hydroxy protecting groups which may be removed without disrupting the remainder of the molecule include acyl and alkylsilyl groups.

For example R^{1′} methoxy is convertible to R^{1′} hydroxy by treatment with lithium and diphenylphosphine (general method described in Ireland et. al. (1973) J. Amer. Chem. Soc., 7829) or HBr. Alkylation of the hydroxy group with a suitable alkyl derivative bearing a leaving group such as halide and a protected amino, piperidyl, amidino or guanidino group or group convertible thereto, yields, after conversion/deprotection, R¹ alkoxy substituted by optionally N-substituted amino, piperidyl, guanidino or amidino.

R³ alkenyl is convertible to hydroxyalkyl by hydroboration using a suitable reagent such as 9-borabicyclo[3.3.1]nonane, epoxidation and reduction or oxymercuration.

R³ 1,2-dihydroxy can be prepared from R^{3′} alkenyl using osmium tetroxide or other reagents well known to those skilled in the art (see Advanced Organic Chemistry (Ed. March, J) (John Wiley and Sons, 1985), p 732-737 and refs. cited therein) or epoxidation followed by hydrolysis (see Advanced Organic Chemistry (Ed. March, J.) (John Wiley and Sons, 1985), p 332,333 and refs. cited therein).

R³ vinyl can be chain extended by standard homologation e.g by conversion to hydroxyethyl followed by oxidation to the aldehyde which is then subjected to a Wittig reaction.

Opening an epoxide R^{3′} group with cyanide anion yields a CH(OH)—CH₂CN group.

Opening an epoxide-containing R^{3′} group with azide anion yields an azide derivative which can be reduced to the amine. Conversion of the amine to a carbamate is followed by ring closure with base to give the 2-oxo-oxazolidinyl containing R³ group.

Substituents on R³ alkyl or alkenyl may be interconverted by conventional methods, for example hydroxy may be derivatised by esterification, acylation or etherification. Hydroxy groups may be converted to halogen, thiol, alkylthio, azido, alkylcarbonyl, amino, aminocarbonyl, oxo, alkylsulphonyl, alkenylsulphonyl or aminosulphonyl by conversion to a leaving group and substitution by the required group, hydrolysis or oxidation as appropriate or reaction with an activated acid, isocyanate or alkoxyisocyanate. Primary and secondary hydroxy groups can be oxidised to an aldehyde or ketone respectively and alkyated with a suitable agent such as an organometallic reagent to give a secondary or tertiary alcohol as appropriate. A carboxylate group may be converted to an hydroxymethyl group by reduction of an ester of this acid with a suitable reducing agent such as lithium aluminium hydride.

Substituted 2-oxo-oxazolidinyl containing R³ groups may be prepared from the corresponding aldehyde by conventional reaction with a glycine anion equivalent, followed by cyclisation of the resulting amino alcohol (M Grauert et al, Ann Chem (1985) 1817, Rozenberg et al, Angew Chem Int Ed Engl (1994) 33(1) 91). The resulting 2-oxo-oxazolidinyl group contains a carboxy group which can be converted to other R¹⁰ groups by standard procedures.

Carboxy groups within R³ may be prepared by Jones' oxidation of the corresponding alcohols CH₂OH using chromium acid and sulphuric acid in water/methanol (E. R. H. Jones et al, J.C.S. 1946,39). Other oxidising agents may be used for this transformation such as sodium periodate catalysed by ruthenium trichloride (G. F. Tutwiler et al, J. Med. Chem., 1987, 30(6), 1094), chromium trioxide-pyridine (G. Just et al, Synth. Commun. 1979, 9(7), 613), potassium permanganate (D. E. Reedich et al, J. Org. Chem., 1985,50(19),3535, and pyridinium chlorochromate (D. Askin et al, Tetrahedron Letters, 1988, 29(3), 277.

The carboxy group may alternatively be formed in a two stage process, with an initial oxidation of the alcohol to the corresponding aldehyde using for instance dimethyl sulphoxide activated with oxalyl chloride (N. Cohen et al, J. Am. Chem. Soc., 1983, 105, 3661) or dicyclohexylcarbodiimide (R. M. Wengler, Angew. Chim. Int. Ed. Eng., 1985, 24(2), 77), or oxidation with tetrapropylammonium perruthenate (Ley et al, J. Chem. Soc. Chem Commun., 1987, 1625). The aldehyde may then be separately oxidised to the corresponding acid using oxidising agents such as silver (II) oxide (R. Grigg et al, J. Chem. Soc. Perkin 1,1983, 1929), potassium permanganate (A. Zurcher, Helv. Chim. Acta., 1987, 70 (7), 1937), sodium periodate catalysed by ruthenium trichloride (T. Sakata et al, Bull. Chem. Soc. Jpn., 1988, 61(6), 2025), pyridinium chlorochromate (R. S. Reddy et al, Synth. Commun., 1988, 18(51), 545) or chromium trioxide (R. M. Coates et al, J. Am. Chem. Soc., 1982, 104, 2198).

An R³ CO₂H group may also be prepared from oxidative cleavage of the corresponding diol, CH(OH)CH₂OH, using sodium periodate catalysed by ruthenium trichloride with an acetontrile-carbontetrachloride-water solvent system (V. S. Martin et al, Tetrahedron Letters, 1988, 29(22), 2701).

R³ groups containing a cyano or carboxy group may also be prepared by conversion of an alcohol to a suitable leaving group such as the corresponding tosylate by reaction with para-toluenesulphonyl chloride (M. R. Bell, J. Med. Chem., 1970, 13, 389), or the iodide using triphenylphosphine, iodine, and imidazole (G. Lange, Synth. Commun., 1990, 20, 1473). The second stage is the displacement of the leaving group with cyanide anion (L A. Paquette et al, J. Org. Chem., 1979, 44 (25), 4603; P. A. Grieco et al, J. Org. Chem., 1988, 53 (16), 3658). Finally acidic hydrolysis of the nitrile group gives the desired acids (H. Rosemeyer et al, Heterocycles, 1985, 23 (10), 2669). The hydrolysis may also be carried out with base e.g. potassium hydroxide (H. Rapoport, J. Org. Chem., 1958, 23, 248) or enzymatically (T. Beard et al, Tetrahedron Asymmetry, 1993, 4 (6), 1085).

Other functional groups in R³ may be obtained by conventional conversions of carboxy or cyano groups.

Tetrazoles are conveniently prepared by reaction of sodium azide with the cyano group (e.g. F. Thomas et al, Bioorg. Med. Chem. Lett., 1996, 6 (6), 631; K. Kubo et al, J. Med. Chem., 1993, 36,2182) or by reaction of azidotri-n-butyl stannane with the cyano group followed by acidic hydrolysis (P. L. Ornstein, J. Org. Chem., 1994, 59, 7682 and J. Med. Chem, 1996, 39 (11), 2219).

The 3-hydroxy-3-cyclobutene-1,2-dion-4-yl group (e.g. R. M. Soll, Bioorg. Med. Chem. Lett., 1993, 3 (4), 757 and W. A. Kinney, J. Med. Chem., 1992, 35 (25), 4720) can be prepared by the following sequence:—(1) a compound where R3 is (CH₂)ₙCHO (n=0, 1, 2) is treated with triethylamine, carbon tetrabromide triphenylphosphine to give initially (CH₂)ₙCH═CBr₂; (2) dehydrobromination of this intermediate to give the corresponding bromoethyne derivative (CH₂)ₙC═CBr (for this 2 stage sequence see D. Grandjean et al, Tetrahedron Letters, 1994, 35 (21), 3529); (3) palladium-catalysed coupling of the bromoethyne with 4-(1-methylethoxy)-3-(tri-n-butylstannyl)cyclobut-3-ene-1,2-dione (Liebeskind et al, J. Org. Chem., 1990, 55, 5359); (4) reduction of the ethyne moity to —CH₂CH₂— under standard conditions of hydrogen and palladium on charcol catalysis (see Howard et al, Tetrahedron, 1980, 36, 171); and finally (4) acidic hydrolysis of the methylethoxyester to generate the corresponding 3-hydroxy-3-cyclobutene-1,2-dione group R. M. Soll, Bioorg. Med. Chem. Lett., 1993, 3 (4), 757).

The tetrazol-5-ylaminocarbonyl group may be prepared from the corresponding carboxylic acid and 2-aminotetrazole by dehydration with standard peptide coupling agents such as 1,1′-carbonyldiimidazole (P. L. Ornstein et al, J. Med Chem, 1996, 39 (11),2232).

The alkyl- and alkenyl-sulphonylcarboxamides are similarly prepared from the corresponding carboxylic acid and the alkyl- or alkenyl-sulphonamide by dehydration with standard peptide coupling agents such as 1,1′-carbonyldiimidazole (P. L. Ornstein et al, J. Med. Chem., 1996, 39 (11), 2232).

The hydroxamic acid groups are prepared from the corresponding acids by standard amide coupling reactions eg N. R. Patel et al, Tetrahedron, 1987, 43 (22), 5375

2,4-thiazolidinedione groups may prepared from the aldehydes by condensation with 2,4-thiazolidinedione and subsequent removal of the olefinic double bond by hydrogenation.

The preparation of 5-oxo-1,2,4-oxadiazoles from nitriles is decribed by Y. Kohara et al, Bioorg. Med. Chem. Lett., 1995, 5(17), 1903.

1,2,4-triazol-5-yl groups may be prepared from the corresponding nitrile by reaction with an alcohol under acid conditions followed by reaction with hydrazine and then an R¹⁰-substituted activated carboxylic acid (see J B Polya in ‘Comprehensive Heterocyclic Chemistry’ Edition 1 p762, Ed A R Katritzky and C W Rees, Pergamon Press, Oxford 1984 and J. J. Ares et al, J. Heterocyclic Chem., 1991, 28(5), 1197).

NH is converted to NR⁴ by conventional means such as amide or sulphonamide formation for compounds where V is CO or SO₂ by methods well known to those skilled in the art or, where V is CH₂, by alkylation with an alkyl halide in the presence of base, reaction with a vinyl derivatives X⁴—X³—CH═CH₂, for example by heating in an alcohol such as ethanol containing an acid such as acetic acid, acylation/reduction or reductive alkylation with an aldehyde.

Examples of R^{4′} convertible to R⁴ include N-protecting groups and —V—CH₂—CO₂H which may be reacted with an amine X⁴—NH₂ in the presence of a coupling agent such as 1-(3-dimethylaminopropyl)-3-ethylcarbodiimide (EDC) to give R⁴—V—CH₂—CO—NH—X⁴. This reaction may be carried out on the compound of formula (V) or after coupling (IV) and (V).

Conventional procedures are used to substitute the piperazine NH with the required group R⁴. For example an oxiranylmethyl group may be introduced by reaction with epichlorohydrin, and the resulting oxirane opened with a suitable amine X⁴—NH₂ to give an R⁴ group of the form —CH₂—CH(OH)—CH₂—NH—X⁴. Cyclisation of the hydroxy and amino (R¹⁴ and R¹³) groups may be effected with phosgene in base. R⁴ groups of the form —CH₂—CH₂—NH—X³—X⁴ may be introduced by alkylation of the piperazine NH. A flexible synthesis involves the introduction of an intermediate group —CH₂—CH₂—NH₂ by treatment with a 2-haloethylcarbamic acid ester followed by reduction. The intermediate group can then be acylated or alkylated as required to introduce the —X³—X⁴ group. R⁴ groups where X³ is O may be prepared from the X⁴-oxirane reacted with an R⁴ CH₂CH₂OH group in the presence of lithium perchlorate.

Interconversion of substituents on the aromatic group X⁴ may be carried out conventionally at any appropriate point in the synthesis. For example pyridine may be oxidised to the the N-oxide with and oxidising agent such as meta-chloroperbenzoic acid, hydrogen peroxide or t-butyl hydroperoxide after protection of any other nitrogen atoms. N-oxides may be rearranged in trifluoroacetic acid to give the hydroxypyridines.

Where one of R³ and R⁶, R⁷, R⁸ or R⁹ contains a carboxy group and the other contains a hydroxy or amino group they may together form a cyclic ester or amide linkage. This linkage may form spontaneously during coupling of the compound of formula (IV) and the piperazine moiety or in the presence of standard peptide coupling agents.

It will be appreciated that under certain circumstances interconvertions may interfere, for example, hydroxy groups in A or B and the piperazine NH will require protection e.g. as a carboxy- or silyl-ester group for hydroxy and as an acyl derivative for piperidine nitrogen, during conversion of R^{1a′}, R^{1′}, R^{3′} or R^{4′}, or during the coupling of the compounds of formulae (IV) and (V).

Compounds of formulae (IV) and (V) are known compounds, (see for example Smith et al, J. Amer. Chem. Soc., 1946, 68, 1301) or prepared analogously, see for example the references cited above for reaction variant (a).

Compounds of formula (IV) where X is CR⁶R⁷SO₂W may be prepared by a route analogous to that of Ahmed El Hadri et al, J. Heterocyclic Chem., 1993, 30(3), 631. Thus compounds of formula (IV) where X is CH₂SO₂OH may be prepared by reacting the corresponding 4-methyl compound with N-bromosuccinimide, followed by treatment with sodium sulfite. The leaving group W may be converted to another leaving group W, e.g. a halogen group, by conventional methods.

The isocyanate of formula (IV) may be prepared conventionally from a 4-amino derivative such as 4-amino-quinoline, and phosgene, or phosgene equivalent (eg triphosgene) or it may be prepared more conveniently from a 4-carboxylic acid by a “one-pot” Curtius Reaction with diphenyl phosphoryl azide (DPPA) [see T. Shiori et al. Chem. Pham. Bull. 35, 2698-2704 (1987)].

The 4-amino derivatives are commercially available or may be prepared by conventional procedures from a corresponding 4-chloro derivative by treatment with ammonia (O. G. Backeberg et. al., J. Chem Soc., 381, 1942) or propylamine hydrochloride (R. Radinov et. al., Synthesis, 886, 1986).

4-Alkenyl compounds of formula (IV) may be prepared by conventional procedures from a corresponding 4-halogeno-derivative by e.g. a Heck synthesis as described in e.g. Organic Reactions, 1982, 2, 345.

4-Halogeno derivatives of compounds of formula (IV) are commercially available, or may be prepared by methods known to those skilled in the art. A 4-chloroquinoline is prepared from the corresponding quinolinone by reaction with phosphorus oxychloride (POCl₃) or phosphorus pentachloride, PCl₅. A 4-chloroquinazoline is prepared from the corresponding quinazolin-4-one by reaction with phosphorus oxychloride (POCl₃) or phosphorus pentachloride PCl₅. A quinazolinone and quinazolines may be prepared by standard routes as described by T. A. Williamson in Heterocyclic Compounds, 6, 324 (1957) Ed. R. C. Elderfield.

4-Carboxy derivatives of compounds of formula (IV) are commercially available or may be prepared by conventional procedures for preparation of carboxy heteroaromatics well known to those skilled in the art. For example, quinazolines may be prepared by standard routes as described by T. A. Williamson in Heterocyclic Compounds, 6, 324 (1957) Ed. R. C. Elderfield. These 4-carboxy derivatives may be activated by conventional means, e.g. by conversion to an acyl halide or anhydride.

Pyridazines may be prepared by routes analogous to those described in Comprehensive Heterocyclic Chemistry, Volume 3, Ed A. J. Boulton and A. McKillop and napthyridines may be prepared by routes analogous to those described in Comprehensive Heterocyclic Chemistry, Volume 2, Ed A. J. Boulton and A. McKillop.

A 4-oxirane derivative of compounds of formula (IV) is conveniently prepared from the 4-carboxylic acid by first conversion to the acid chloride with oxalyl chloride and then reaction with trimethylsilyldiazomethane to give the diazoketone derivative. Subsequent reaction with SM hydrochloric acid gives the chloromethylketone. Reduction with sodium borohydride in aqueous methanol gives the chlorohydrin which undergoes ring closure to afford the epoxide on treatment with base, e.g. potassium hydroxide in ethanol-tetrahydrofuran.

Alternatively and preferably, 4-oxirane derivatives can be prepared from bromomethyl ketones which can be obtained from 4-hydroxy compounds by other routes well known to those skilled in he art. For example, hydroxy compounds can be converted to the corresponding 4-trifluoromethanesulphonates by reaction with trifluoromethanesulphonic anhydride under standard conditions (see K. Ritter, Synthesis, 1993, 735). Conversion into the corresponding butyloxyvinyl ethers can be achieved by a Heck reaction with butyl vinyl ether under palladium catalysis according to the procedure of W. Cabri et al, J. Org. Chem, 1992, 57 (5), 1481. (Alternatively, the same intermediates can be attained by Stille coupling of the trifluoromethanesulphonates or the analaogous chloro derivatives with (1-ethoxyvinyl)tributyl tin, T. R. Kelly, J. Org. Chem., 1996, 61, 4623.) The alkyloxyvinyl ethers are then converted into the corresponding bromomethylketones by treatment with N-bromosuccinimide in aqueous tetrahydrofuran in a similar manner to the procedures of J. F. W. Keana, J. Org. Chem., 1983, 48, 3621 and T. R Kelly, J. Org. Chem., 1996, 61, 4623.

The 4-hydroxyderivatives can be prepared from an aminoaromatic by reaction with methylpropiolate and subsequent cyclisation, analogous to the method described in N. E. Heindel et al, J. Het. Chem., 1969, 6, 77. For example, 5-amino-2-methoxy pyridine can be converted to 4-hydroxy-6-methoxy-[1,5]naphthyridine using this method.

If a chiral reducing agent such as (+) or (−)-B-chlorodiisopinocamphenylborane [‘DIP-chloride’ ] is substituted for sodium borohydride, the prochiral chloromethylketone is converted into the chiral chlorohydrin with ee values generally 85-95% [see C. Bolm et al, Chem. Ber. 125, 1169-1190, (1992)]. Recrystallisation of the chiral epoxide gives material in the mother liquor with enhanced optical purity (typically ee 95%).

The (R)-epoxide, when reacted with a piperazine derivative gives ethanolamine compounds as single diastereomers with (R)-stereochemistry at the benzylic position.

Alternatively, the epoxide may be prepared from the 4-carboxaldehyde by a Wittig approach using trimethylsulfonium iodide [see G. A. Epling and K-Y Lin, J. Het. Chem., 1987, 24, 853-857], or by epoxidation of a 4-vinyl derivative. 4-Hydroxy-1,5-naphthyridines can be prepared from 3-aminopyridine derivatives by reaction with diethyl ethoxymethylene malonate to produce the 4-hydroxy-3-carboxylic acid ester derivative with subsequent hydrolysis to the acid, followed by thermal decarboxylation in quinoline (as for example described for 4-Hydroxy[1,5]naphthyridine-3-carboxylic acid, J. T. Adams et al., J. Amer. Chem. Soc., 1946, 68, 1317). A 4-hydroxy-[1,5]naphthyridine can be converted to the 4-chloro derivative by heating in phosphorus oxychloride, or to the 4-methanesulphonyloxy or 4-trifluoromethanesulphonyloxy derivative by reaction with methanesulphonyl chloride or trifluoromethanesulphonic anhydride, respectively, in the presence of an organic base. A 4-amino 1,5-naphthyridine can be obtained from the 4-chloro, 4-methanesulphonyloxy or 4-trifluoromethanesulphonyloxy derivative by reaction with n-propylamine in pyridine.

Similarly, 6-methoxy-1,5-naphthyridine derivatives can be prepared from 3-amino-6-methoxypyridine.

1,5-Naphthyridines may be prepared by other methods well known to those skilled in the art (for examples see P. A. Lowe in “Comprehensive Heterocyclic Chemistry” Volume 2, p581-627, Ed A. R Katritzky and C. W. Rees, Pergamon Press, Oxford, 1984).

The 4-hydroxy and 4-amino-cinnolines may be prepared following methods well known to those skilled in the art [see A. R. Osborn and K. Schofield, J. Chem. Soc. 2100 (1955)]. For example, a 2-aminoacetopheneone is diazotised with sodium nitrite and acid to produce the 4-hydroxycinnoline with conversion to chloro and amino derivatives as described for 1,5-naphthyridines.

The substituted piperazines of formula (V) are either commercially available or may be synthesised by hydrogenation of the corresponding pyrazines (eg von E. Felder et al. Helv. Chim. Acta 33, 888-896 (1960)], or by diborane reduction of a suitable lactam [eg H. L. Larkins et al. Tet. Lett. 27, 2721-2724 (1986)].

Chiral piperazines may be prepared from chiral 2-(S)- and 2-(R)-piperazinecarboxylic acid. Racemic piperazine-2-carboxylic acid (commercially available) may be resolved by crystallisation of the (S)- and (R)-dicamphor-10-sulfonic acid salts [following the general method of K. Stingl et al. Tet. Asymmetry 8, 979-982 (1997)-described for preparation of 2-(S)-piperazinecarboxylic acid].

Piperazine-2-carboxylic acid may be differentially protected [following the procedure of C. F. Bigge et al. Tet. Lett. 30, 5193-5196 (1989)] by first reacting with 2-(t-butoxycarbonyloxyimino)-2-phenylacetonitrile which selectively reacts on N-4, and then by reacting with benzylchloroformate which reacts on N-1. The 2-carboxylic acid is then methylated (conveniently with TMS-diazomethane). Hydrogenation (over Pd/C) then removes the carbobenzyloxy group, which may be alkylated or acylated with the required R⁴ group as described above for the conversion of R^{4′}═H to R⁴. Reaction with trifluoroacetic acid (optionally in dichloromethane) removes the N-4-butoxycarbonyloxy group to afford the required 4-H piperazine.

The chiral piperazine-2-carboxylic acids may be elaborated to various derivatives, for example, an Arndt-Eistert procedure (involving silver salt mediated rearrangement of a diazoketone) will give chiral 2-acetic acid derivatives (initially via the methyl ester). Reduction of the intermediate ester with standard reducing agents such as lithium aluminium hydride will produce a hydroxymethyl derivative.

Compounds of formula (V) where n=1 may be prepared from the compound where n=0 by homologation eg starting from a compound of formula (V) where Y═CO₂H.

R⁴-halides, vinyl derivatives X⁴—X³—CH═CH₂, acyl derivative X⁴—X³—X²—X¹—COW and X⁴—X³—X²—X¹—SO₂W or aldehydes X⁴—X³—X²—X¹—CHO are commercially available or are prepared conventionally. The aldehydes may be prepared by partial reduction of the corresponding ester (see Reductions by the Alumino- and Borohydrides in Organic Synthesis, 2nd ed., Wiley, N.Y., 1997; JOC, 3197, 1984; Org. Synth. Coll., 102, 1990; 136, 1998; JOC, 4260, 1990; TL, 995, 1988; JOC, 1721, 1999; Liebigs Ann./Recl., 2385, 1997; JOC, 5486, 1987) with lithium aluminium hydride or diisobutylaluminium hydride or more preferably by reduction to the alcohol, with lithium aluminium hydride or sodium borohydride, followed by oxidation to the aldehyde with manganese (II) dioxide. The aldehydes may also be prepared from carboxylic acids by conversion to a mixed anhydride for example by reaction with isobutyl chloroformate followed by reduction with sodium borohydride (R. J. Alabaster et al., Synthesis, 598, 1989) to give the benzylic alcohols and then oxidation with a standard oxidising agent such as pyridinium dichromate or manganese (II) dioxide. Acyl derivative may be prepared by activation of the corresponding ester. R⁴-halides such as bromides may be prepared from the alcohol R⁴OH by reaction with phosphorus tribromide in DCM/triethylamine. Where X² is CO and X³ is NR¹³ the R⁴-halide may be prepared by coupling an X⁴—NH₂ amine and bromopropionyl bromide. Vinyl derivatives X⁴—X³CH═CH₂ may be prepared from the corresponding diethylaminoethyl derivative by quaternisation with dimethyl sulphate and heating, resulting in elimination of the charged amino group. The diethylaminoethyl derivative may be prepared from another ethyl derivative eg the hydroxyethyl by conventional amine formation. Alternatively, it may be prepared from a methyl derivative by condensation with diethylamine and formaldehyde.

Conversions of R^{1a′}, R^{1′}, R^{3′} and R^{4′} may be carried out on the intermediates of formulae (IV), (V) and (Vb) prior to their reaction to produce compounds of formula (I) in the same way as described above for conversions after their reaction.

The pharmaceutical compositions of the invention include those in a form adapted for oral, topical or parenteral use and may be used for the treatment of bacterial infection in mammals including humans.

The antibiotic compounds according to the invention may be formulated for administration in any convenient way for use in human or veterinary medicine, by analogy with other antibiotics.

The composition may be formulated for administration by any route, such as oral, topical or parenteral. The compositions may be in the form of tablets, capsules, powders, granules, lozenges, creams or liquid preparations, such as oral or sterile parenteral solutions or suspensions.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrollidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilizing the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compositions may contain from 0.1% by weight, preferably from 10-60% by weight, of the active material, depending on the method of administration. Where the compositions comprise dosage units, each unit will preferably contain from 50-500 mg of the active ingredient. The dosage as employed for adult human treatment will preferably range from 100 to 3000 mg per day, for instance 1500 mg per day depending on the route and frequency of administration. Such a dosage corresponds to 1.5 to 50 mg/kg per day. Suitably the dosage is from 5 to 20 mg/kg per day.

No toxicological effects are indicated when a compound of formula (I) or a pharmaceutically acceptable salt or in vivo hydrolysable ester thereof is administered in the above-mentioned dosage range.

The compound of formula (I) may be the sole therapeutic agent in the compositions of the invention or a combination with other antibiotics or with a P-lactamase inhibitor may be employed.

Compounds of formula (I) are active against a wide range of organisms including both Gram-negative and Gram-positive organisms.

The following examples illustrate the preparation of certain compounds of formula (I) and the activity of certain compounds of formula (I) against various bacterial organisms.

### EXAMPLES

### Example 1

### (R)-1-(3,5-Difluoro-phenylamino)-3-{4-[(R)-2-hydroxy-2-(6-methoxy-quinolin-4-yl)-ethyl]-piperazin-1-yl}-propan-2-ol Dioxalate Salt

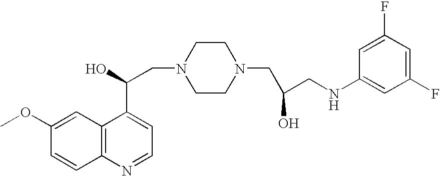

(a) 6-Methoxyquinoline-4-carboxylic Acid

The title compound was prepared by modification of the procedure described by W. E. Daering and J. D. Chanley, J. Amer. Chem. Soc., 1946, 68, 586. A mixture of Quinine (225 g, 0.70 mol), tert-butanol (1 litre) and water (10 ml) was treated with potassium tert-butoxide (170 g, 1.5 mol). The mixture was stirred at 30° C., while air was bubbled through for 3 days. The mixture was diluted with diethyl ether and water and the layers separated. The aqueous phase was extracted with ethyl acetate. The combined diethyl ether and ethyl acetate extracts were dried over magnesium sulfate and evaporated to give recovered starting material (approximately 10 g). The aqueous phase was acidified to pH 5 with 5M hydrochloric acid. The precipitate was collected by filtration, washed with water and methanol, then dried to give 6-methoxyquinoline-4-carboxylic acid as a yellow solid (64.6 g, 46%).

δH (d-6 DMSO), 6.23-5.95 (1H, m), 5.34-5.06 (2H, m), 3.37-2.92 (5H, m), 2.70 (1H, m), 2.38-2.15 (3H, m), 1.94-1.52 (2H, m).

(b) (R)-2-(6-Methoxyquinolin-4-yl)oxirane

A solution of 6-methoxyquinoline-4-carboxylic acid (10 g) in dichloromethane was heated under reflux with oxalyl chloride (5 ml) and N,N′-dimethylformamide (2 drops) for 1 hour and evaporated to dryness. The residue, in dichloromethane (100 ml) was treated with a 2M solution of trimethylsilyldiazomethane in hexane (50 ml) and stirred at room temperature for 18 hours. 5M Hydrochloric acid (150 ml) was added and the solution was stirred at room temperature for 3 hours. It was basified with sodium carbonate solution, extracted with ethyl acetate and chromatographed on silica gel eluting with ethyl acetate-hexane to give the chloromethyl ketone (4.2 g). A batch of the chloromethyl ketone (20 g) was reduced with (+)-B-chlorodiisopinocamphenylborane (40 g) in dichloromethane (400 ml) at room temperature for 18 hours followed by treatment with diethanolamine (30 g) for 3 hours. The product was chromatographed on silica gel eluting with ethyl acetate-hexane to give the chloroalcohol (16.8 g), which was dissolved in tetrahydrofuran (100 ml) and reacted with sodium hydroxide (2.6 g) in water (13 ml) for 1.5 hours. The reaction mixture was evaporated to dryness and chromatographed on silica gel eluting with ethyl acetate/hexane to give the title compound as a solid (10.4 g) (84% ee by chiral HPLC).

Recrystallisation from ether-pentane gave mother-liquor (7.0 g) (90% ee).

MS (+ve ion electrospray) m/z 202 (MH+)

The absolute stereochemistry was defined to be (R) by an NMR study on the Mosher's esters derived from the product obtained by reaction with 1-tert-butylpiperazine.

(c) 4-[(R)-2-Hydroxy-2-(6-methoxy-quinolin-4-yl)-ethyl]-piperazine-1-carboxylic Acid Tert-Butyl Ester

(R)-2-(6-Methoxyquinolin-4-yl)oxirane (4.30 g, 21.37 mmol) was dissolved in acetonitrile (30 mL). To the solution was added piperazine-1-carboxylic acid tert-butyl ester (7.17 g, 38.47 mmol) and lithium perchlorate (2.27 g, 106.4 mmol). The resulting slurry was stirred at room temperature for 10 hours and then concentrated in vacuo. The residue was partitioned between ethyl acetate and water and the organic phase dried over magnesium sulfate. Concentration in vacuo afforded a colourless oil which was subjected to purification by column chromatography on silica gel using a dichloromethane/methanol gradient. This provided the desired compound as a colourless oil (7.65 g, 92%).

MS (APCI+) m/z 388 (MH+).

(d) (R)-1-(6-Methoxy-quinolin-4-yl)-2-piperazin-1-yl-ethanol

4-[(R)-2-Hydroxy-2-(6-methoxy-quinolin-4-yl)-ethyl]-piperazine-1-carboxylic acid tert-butyl ester (3.50 g) was dissolved in dichloromethane (10 mL) and trifluoroacetic acid (10 mL) was added. The resulting solution was stirred at room temperature for 5 hours and then concentrated in vacuo. The residue was purified on silica gel, eluting with methanol and dichloromethane. This provided the desired compound which was used without further purification.

MS (APCI+) m/z 288 (MH+).

(e) (R)-1-(6-Methoxy-quinolin-4-yl)-2-(4-(S)-1-oxiranylmethyl-piperazin-1-yl)-ethanol

(R)-1-(6-Methoxy-quinolin-4-yl)-2-piperazin-1-yl-ethanol (1.80 g, 2.50 mmol) was dissolved in a 1:1 mixture of tetrahydrofuran and water (5 mL). (R)-epichlorohydrin (280 mg, 3.01 mmol) was added and the solution stirred for 5 hours at room temperature. The solution was concentrated in vacuo and the residue partitioned between ethyl acetate and saturated aqueous sodium bicarbonate. The organic phase was dried over magnesium sulfate and concentrated in vacuo. The residue was subjected to chromatography on silica gel with methanol and dichloromethane to provide a colourless oil (240 mg, 11%).

MS (APCI+) m/z 344 (MH+).

(f) Title Compound

(R)-1-(6-Methoxy-quinolin-4-yl)-2-(4-(S)-1-oxiranylmethyl-piperazin-1-yl)-ethanol (117 mg, 0.341 mmol) was dissolved in ethanol (3 mL) and then 3,5-difluoroaniline (88 mg, 0.682 mmol) was added. The resulting solution was heated to 80° C. for 10 hours. The solution was concentrated in vacuo and the residue subjected to chromatography on silica gel using methanol and dichloromethane as eluant. This afforded the desired compound as a colourless oil (19 mg, 12%).

δH (CD₃OD, 250 MHz) 8.65-8.23 (1H, d), 7.93-7.89 (1H, m), 7.67-7.87 (1H, d), 7.41-7.37 (2H, m), 6.21-6.14 (2H, m), 6.06-5.97 (1H, m), 5.62-5.46 (1H, dd), 3.93 (3H, s), 3.89-3.85, 3.22-3.00, 2.90-2.45 (15H, m).

MS (APCI+) m/z 473 (MH+).

A solution of the oil (19 mg) in dichloromethane (1 mL) was added to oxalic acid (7 mg) in diethyl ether (10 mL) to generate the dioxalate salt. The title compound was isolated by centrifugation, washing with diethyl ether and subsequent drying in vacuo.

### Example 2

### (R/S)-1-(3,5-Difluoro-phenylamino)-3-{4-[(R)-2-hydroxy-2-(6-methoxy-quinolin-4-yl)-ethyl]-piperazin-1-yl}-propan-2-ol

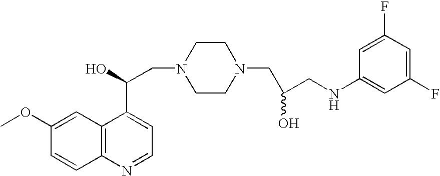

The title compound was prepared in the same manner as (R)-1-(3,5-Difluoro-phenylamino)-3-{4-[(R)-2-hydroxy-2-(6-methoxy-quinolin-4-yl)-ethyl]-piperazin-1-yl}-propan-2-ol but starting from racemic epichlorohydrin.

MS (APCI+) m/z 473 (MH+).

### Example 3

### 3-(3,5-Difluoro-phenyl)-5-{4-[(R)-2-hydroxy-2-(6-methoxy-quinolin-4-yl)-ethyl]-piperazin-1-ylmethyl}-oxazolidin-2-one Dimesylate Salt

(R/S)-1-(3,5-Difluoro-phenylamino)-3-{4-[(R)-2-hydroxy-2-(6-methoxy-quinolin-4-yl)-ethyl]-piperazin-1-yl}-propan-2-ol (Example 2) was dissolved in dichloromethane (1 mL) and triethylamine (0.02 mL, 0.16 mmol) was added followed by phosgene (20% in toluene) (0.04 mL, 0.08 mmol). The solution was stirred at room temperature for 10 hours and then concentrated in vacuo. The residue was partitioned between ethyl acetate and saturated aqueous sodium bicarbonate. The organic phase was dried over magnesium sulfate and the volatiles removed under reduced pressure. The resulting oil was purified on silica gel using methanol and dichloromethane as eluant. This afforded the desired compound as a yellow oil (6 mg, 15%).

δH (CD₃OD, 250 MHz) 8.79-8.77 (1H, m), 8.07-8.03 (1H, m), 7.65-7.63 (1H, m), 7.37-7.36 (2H, m), 7.18-7.16 (2H, m), 6.63-6.56 (1H, m), 5.48-5.44 (1H, m), 4.81 (1H, m), 3.94 (3H, s), 4.04-3.65, 2.95-2.50 (14H, m).

MS (APCI+) m/z 499 (MH+).

A solution of the oil (6 mg) in dichloromethane (1 mL) was added to a 0.1M solution of methanesulfonic acid in diethyl ether (0.24 mL) in more diethyl ether (10 mL) to generate the dimesylate salt. The title compound was isolated by centrifuigation, washing with diethyl ether and subsequent drying in vacuo.

### Example 4

### N-(2-{4-[(R)-2-Hydroxy-2-(6-methoxy-quinolin-4-yl)-ethyl]-piperazin-1-yl}-ethyl)-benzamide Dioxalate Salt

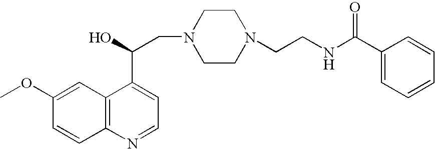

(a) (2-{4-[(R)-2-Hydroxy-2-(6-methoxy-quinolin-4-yl)-ethyl]-piperazin-1-yl}-ethyl)-carbamic Acid Benzyl Ester

(R)-1-(6-Methoxy-quinolin-4-yl)-2-piperazin-1-yl-ethanol (Example 1d) (2.60 g, 9.04 mmol) was slurried in benzene (20 mL) and N,N′-dimethylformamide (6 mL). Potassium carbonate (1.87 g, 13.57 mmol) was added followed by (2-bromo-ethyl)-carbamic acid benzyl ester (3.50 g, 13.57 mmol). The resulting slurry was heated to 70° C. for 10 hours under argon. Concentration in vacuo was followed by extraction into ethyl acetate then drying of the organic phase over magnesium sulfate. Concentration in vacuo provided an oil which was subjected to chromatography on silica gel. This afforded the desired product (1.01 g, 24%).

MS (APCI+) m/z 465 (MH+).

(b) (R)-2-[4-(2-Amino-ethyl)-piperazin-1-yl]-1-(6-methoxy-quinolin-4-yl)-ethanol

(2-{4-[(R)-2-Hydroxy-2-(6-methoxy-quinolin-4-yl)-ethyl]-piperazin-1-yl}-ethyl)-carbamic acid benzyl ester (1.00 g, 2.16 mmol) was dissolved in methanol (10 mL) and treated with 10% Pd—C catalyst (20 mg) then hydrogenated under 1 atmosphere of hydrogen gas at room temperature for 10 hours. The catalyst was removed by filtration through celite® washing with methanol (20 mL) and the filtrate concentrated in vacuo. This afforded the desired product (583 mg, 82%) which was used without further purification.

MS (APCI+) m/z 331 (MH+).

(c) Title Compound

(R)-2-[4-(2-Amino-ethyl)-piperazin-1-yl]-1-(6-methoxy-quinolin-4-yl)-ethanol (55 mg, 0.166 mmol) was dissolved in N,N′-dimethylformamide (0.5 mL). 1-(3-Dimethylaminopropyl)-3-ethylcarbodiimide hydrochloride (48 mg, 0.249 mmol), benzoic acid (30 mg, 0.249 mmol) and triethylamine (0.07 mL, 0.498 mmol) were then added and the resulting slurry stirred at room temperature for 10 hours. The solvent was removed in vacuo and the residue subjected to column chromatography on silica gel. This afforded the desired amide (24 mg, 33%).

δH (CD₃OD, 250 MHz), 8.68-8.66 (1H, d), 7.97-7.41 (9H, m), 5.68-5.63 (1H, dd), 3.96 (3H, s), 3.61-3.56 (2H, m), 2.87-2.71 (12H, m).

MS (APCI+) m/z 436 (MH+).

A solution of the oil (24 mg) in dichloromethane (1 mL) was added to oxalic acid (10 mg) in diethyl ether (10 mL) to generate the dioxalate salt. The title compound was isolated by centrifugation, washing with diethyl ether and subsequent drying in vacuo.

### Example 5

### 3,5-Difluoro-N-(2-{4-[(R)-2-hydroxy-2-(6-methoxy-quinolin-4-yl)-ethyl]-piperazin-1-yl}-ethyl)-benzamide Dioxalate Salt

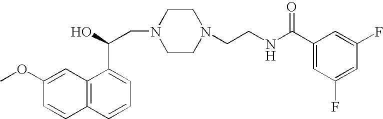

The title compound was prepared in the same manner as Example 4. This afforded the desired compound (33 mg, 42%).

MS (APCI+) m/z 471 (M+).

The corresponding dioxalate salt was prepared in the same manner as Example 1.

### Example 6

### Pyridine-2-carboxylic acid (2-{4-[(R)-2-hydroxy-2-(6-methoxy-quinolin-4-yl)-ethyl]-piperazin-1-yl}-ethyl)-amide Dioxalate Salt

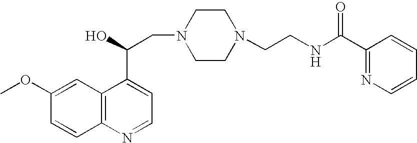

The title compound was prepared in the same manner as Example 4. This afforded the desired compound (11 mg, 15%).

MS (APCI+) m/z 436 (MH+).

The corresponding dioxalate salt was prepared in the same manner as Example 1.

### Example 7

### Thiophene-2-carboxylic acid (2-{14[(R)-2-hydroxy-2-(6-methoxy-quinolin-4-yl)-ethyl]-piperazin-1-yl}-ethyl)-amide Dioxalate Salt

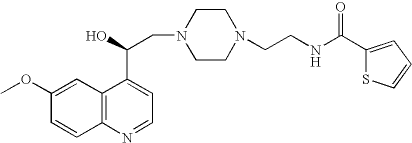

The title compound was prepared in the same manner as Example 4. This afforded the desired compound (13 mg, 18%).

MS (APCI+) m/z 441 (MH+).

The corresponding dioxalate salt was prepared in the same manner as Example 1.

### Example 8

### Furan-2-carboxylic acid (2-{4-[R)-2-hydroxy-2-(6-methoxy-quinolin-4-yl)-ethyl]-piperazin-1-yl}-ethyl)-amide Dioxalate Salt

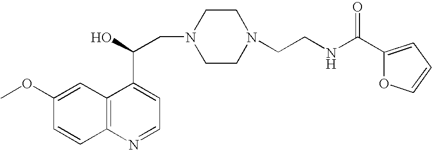

The title compound was prepared in the same manner as Example 4. This afforded the desired compound (24 mg, 34%).

MS (APCI+) m/z 425 (M+).

The corresponding dioxalate salt was prepared in the same manner as Example 1.

### Example 9

### 3-Cyano-N-(2-{4-[(R)-2-hydroxy-2-(6-methoxy-quinolin-4-yl)-ethyl]-piperazin-1-yl}-ethyl)-benzamide Dioxalate Salt

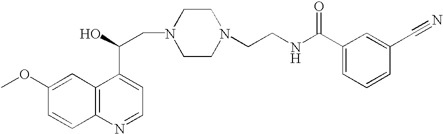

The title compound was prepared in the same manner as Example 4. This afforded the desired compound (25 mg, 33%).

MS (APCI+) m/z 460 (MH+).

The corresponding dioxalate salt was prepared in the same manner as Example 1.

### Example 10

### N-(2-{4-[(R)-2-Hydroxy-2-(6-methoxy-quinolin-4-yl)-ethyl]-piperazin-1-yl}-ethyl)-trifluoromethyl-benzamide Dioxalate Salt

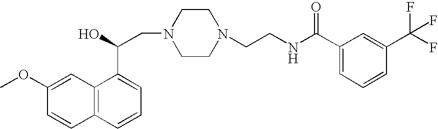

The title compound was prepared in the same manner as Example 4. This afforded the desired compound (25 mg, 30%).

MS (APCI+) m/z 503 (MH+).

The corresponding dioxalate salt was prepared in the same manner as Example 1.

### Example 11

### (E)-1-{4-[(R)-2-Hydroxy-2-(6-methoxy-quinolin-4-yl)-ethyl]-piperazin-1-yl}4-phenyl-but-3-en-1-one Dioxalate Salt

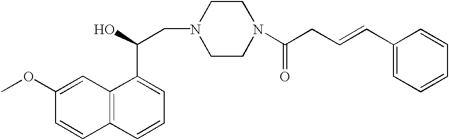

(R)-1-(6-Methoxy-quinolin-4-yl)-2-piperazin-1-yl-ethanol (Example 1 d) (275 mg, 0.96 mol) was dissolved in N,N′-dimethylformamide (2 mL). 1-(3-Dimethylaminopropyl)-3-ethylcarbodiimide hydrochloride (276 mg, 1.44 mmol), styrylacetic acid (233 mg, 1.44 mmol) and triethylamine (0.40 mL, 2.87 mmol) were then added and the resulting slurry stirred at room temperature for 10 hours. The solvent was removed in vacuo and the residue subjected to column chromatography on silica gel. This afforded the desired amide (56 mg, 14%).

δH (CDCl₃, 250 MHz), 8.79-8.77 (1H, d), 8.08-8.04 (1H, d), 7.65-7.63 (1H, d), 7.40-7.13 (7H, m), 6.56-6.26 (2H, m), 5.51-5.46 (1H, dd), 3.90 (3H, s), 3.90-3.30, 2.88-2.48 (12H, m).

MS (APCI+) m/z 432 (MH+).

A solution of the oil (18.5 mg) in dichloromethane (1 mL) was added to oxalic acid (8 mg) in diethyl ether (10 mL) to generate the dioxalate salt. The title compound was isolated by centrifugation, washing with diethyl ether and subsequent drying in vacuo.

### Example 12

### (R)-1-(6-Methoxy-quinolin-4-yl)-2-[4-(4-phenyl-butyl)piperazin-1-yl]-ethanol Dioxalate (a) 4-Phenyl-butan-1-al

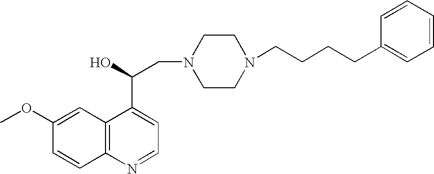

4-Phenyl-butan-1-ol (1.50 g, 9.99 mmol) was dissolved dichloromethane (10 mL) and pyridinium chlorochromate (3.45 g, 15.98 mmol) was added. The slurry was stirred at room temperature for 1 hour and then filtered through silica gel eluting with an ethyl acetate/hexanes gradient. The desired compound was isolated as a yellow oil.

MS (APCI+) m/z 147 (M+−1).

(b) Title Compound

(R)-1-(6-Methoxy-quinolin-4-yl)-2-piperazin-1-yl-ethanol (Example 1 d) (410 mg, 1.42 mmol) was dissolved in dichloromethane (2 mL) and methanol (0.5 mL) and 4-phenyl-butan-1-al (253 mg, 1.71 mmol) was added. The solution was stirred at room temperature over 4 Å molecular seives for 10 hours and then sodium triacetoxyborohydride (454 mg, 2.14 mmol) was added. The resulting slurry was stirred at room temperature for a further 5 hours. The volatiles were then removed in vacuo and the residue subjected to purification by column chromatography on silica gel eluting with a methanol/dichloromethane gradient. This provided the desired compound as a yellow oil (271 mg, 45%)

δH (CDCl₃, 250 MHz) 8.76-8.75 (1H, d), 8.05-8.01 (1H, d), 7.65-7.63 (1H, d), 7.39-7.16 (7H, m), 5.47-5.41(1H, dd), 3.91 (3H, s), 2.87-2.49 (14H, m), 1.71-1.48 (4H, m). MS (APCI+) m/z 420 (MH+).

A solution of the oil (41 mg) in dichloromethane (1 mL) was added to oxalic acid (18.5 mg) in diethyl ether (10 mL) to generate the dioxalate salt. The title compound was isolated by centrifugation, washing with diethyl ether and subsequent drying in vacuo.

### Example 13

### (S)-1-(6-Methoxy-quinolin-4-yl)-2-[4(4-phenyl-butyl)-piperazin-1-yl]-ethylamine Trioxalate Salt

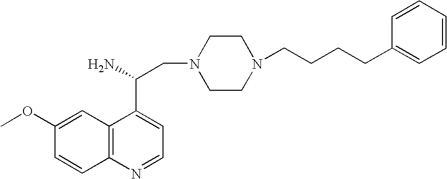

(a) 4-{(S)-1-Azido-2-[4-(4-phenyl-butyl)-piperazin-1-yl]-ethyl}-6-methoxy-quinoline

(R)-1-(6-Methoxy-quinolin-4-yl)-2-piperazin-1-yl-ethanol (Example 1 d) (148 mg, 0.352 mmol) was slurried in a mixture of toluene (3.5 mL) and dichloromethane (0.5 mL). Diphenyl phosphoryl azide (0.23 mL, 1.06 mmol) was added followed by 1,8-diazabicyclo[5.4.0]undec-7-ene (0.16 mL, 1.06 mmol). The slurry was stirred for 10 hours at room temperature after which time it was concentrated under reduced pressure. The residue was subjected to purification on silica gel eluting with a gradient of dichloromethane and methanol. This afforded the desired product as a yellow oil (37 mg, 24%).

MS (APCI+) m/z 445 (MH+).

(b) Title compound

4-{(S)-1-Azido-2-[4-(4-phenyl-butyl)-piperazin-1-yl]-ethyl}-6-methoxy-quinoline was dissolved in ethanol (2 mL) and 10% palladium on carbon (20 mg) was added. The slurry was hydrogenated under one atmosphere of hydrogen at room temperature. After 10 hours the catalyst was removed by filtration through celite® washing with ethanol. The filtrate was concentrated under reduced pressure and the residue purified on silica gel using a dichloromethane/methanol gradient. This provided the desired compound as a colourless oil (10 mg, 29%).

δH (CD₃OD, 250 MHz), 8.68-8.66 (1H, d), 7.96-7.93 (1H, d), 7.66-7.65 (1H, d), 7.46-7.05 (7H, m), 5.01-4.96 (1H, dd), 3.97 (3H, s), 2.79-2.34 (14H, m), 1.64-1.59 (4H, m). MS (APCI+) m/z 419 (MH+).

A solution of the oil (10 mg) in dichloromethane (1 mL) was added to oxalic acid (6.5 mg) in diethyl ether (10 mL) to generate the trioxalate salt. The title compound was isolated by centrifugation, washing with diethyl ether and subsequent drying in vacuo.

### Example 14

### N-(3,5-Difluoro-phenyl)-3-{4-[(R)-2-hydroxy-2-(6-methoxy-quinolin-4-yl)-ethyl]-piperazin-1-yl}-propionamide Dioxalate Salt

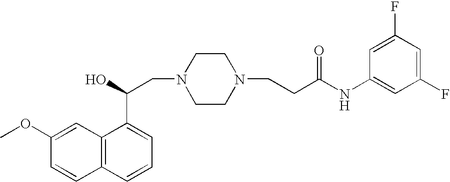

3-{4-[(R)-2-Hydroxy-2-(6-methoxy-quinolin-4-yl)-ethyl]-piperazin-1-yl}-propionic acid tert-butyl ester (Example 1c) (800 mg, 1.93 mmol) was dissolved in dichloromethane (10 mL) and trifluoroacetic acid (10 mL) was added. The resulting solution was stirred at room temperature for 2 hours and then concentrated in vacuo. The resulting TFA salt (121 mg) was dissolved in N,N′-dimethylformamide (1 mL). To the solution was added 3,5-difluoroaniline (40 mg, 0.31 mmol) and 1-(3-dimethylaminopropyl)-3-ethylcarbodiimide hydrochloride (60 mg, 0.311 mmol). This was followed by the addition of triethylamine (0.09 mL, 0.62 mmol). The solution was stirred at room temperature for 5 hours and then concentrated in vacuo. The residue was purified on neutral alumina using a dichloromethane/methanol gradient. This afforded the desired compound as a colourless oil (16 mg, 16%).

δH (CD₃OD, 250 MHz), 8.49-8.47 (1H, d), 7.77-7.73 (1H, m), 7.51-7.49 (1H, d), 7.25-7.22 (3H, m), 7.10-7.01 (1H, m), 6.50-6.41 (1H, m), 5.49-5.46 (1H, m), 3.77 (3H, s), 3.20-2.40 (14H).

MS (APCI+) m/z 471 (MH+).

A solution of the oil (16 mg) in dichloromethane (1 mL) was added to oxalic acid (6 mg) in diethyl ether (10 mL) to generate the dioxalate salt. The title compound was isolated by centrifugation, washing with diethyl ether and subsequent drying in vacuo.

### Example 15

### 1-{4-[(R)-2-Hydroxy-2-(6-methoxy-quinolin-4-yl)-ethyl]-piperazin-1-yl}-3-phenoxy-propan-2-ol Dioxalate Salt

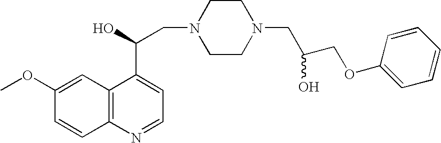

(R)-1-(6-Methoxy-quinolinyl)-2-piperazin-1-yl-ethanol (Example 1d) (258 mg, 0.899 mmol) was dissolved in acetonitrile (4 mL). To the resulting solution was added 2-phenoxymethyl oxirane (150 mg, 0.989 mmol) and lithium perchlorate (105 mg, 0.989 mmol). The solution was stirred at 50° C. for 10 hours and then concentrated under reduced pressure. The residue was subjected to purification by column chromatography on silica gel using a dichloromethane/methanol gradient. This allowed isolation of the desired product (63 mg, 16%).

δH (CD₃OD, 250 MHz), 8.70-8.69 (1H, d), 7.98-7.93 (2H, m), 7.75-7.73 (1H, m), 7.45-6.94 (6H, m), 5.75-5.72 (1H, m), 4.654.60 (1H, m), 3.92 (3H, s), 3.50-2.75 (14H, m).

MS (APCI+) m/z 438 (MH+).

A solution of the oil (43 mg) in dichloromethane (1 mL) was added to oxalic acid (18 mg) in diethyl ether (10 mL) to generate the dioxalate salt. The title compound was isolated by centrifugation, washing with diethyl ether and subsequent drying in vacuo.

### Example 16

### 3-(3-Fluoro-phenyl)-5-{4[(R)-2-hydroxy-2-(6-methoxy-quinolin-4-yl)ethyl]-piperazin-1-ylmethyl}-oxazolidin-2-one Dimesylate

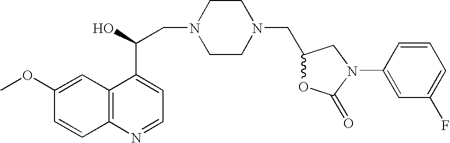

(a) (R/S)-5-Bromomethyl-3-(3-fluoro-phenyl)-oxazolidin-2-one

Lithium bromide (32 mg, 0.362 mmol) and tri-n-butyl phosphine (111 mg, 0.507 mmol) were heated in xylene (5 mL) to 140° C. for 1 hour. (R/S)-2-Bromomethyl-oxirane (0.62 mL, 7.25 mmol) and 3-fluorophenyl isocyanate (0.993 g, 7.25 mmol) were added and the mixture was heated to 140° C. for 3 hours and then partitioned between ethyl acetate and brine. The organic phase was dried over magnesium sulfate and concentrated in vacuo. The residue was subjected to purification by column chromatography on silica gel using an ethyl acetate/hexanes gradient. This afforded the desired compound as a colourless oil (1.75 g, 88%).

(b) (R/S)-4-(2-Oxo-3-phenyl-oxazolidin-5-ylmethyl)-piperazine-1-carboxylic Acid Tert-Butyl Ester

(R/S)-5-Bromomethyl-3-(3-fluoro-phenyl)-oxazolidin-2-one (0.894 g, 3.251 mmol) was dissolved in N,N′-dimethylformamide (10 mL) and piperazine-1-carboxylic acid benzyl ester (0.715 g, 3.251 mmol) and diisopropyl ethylamine (0.68 mL, 3.90 mmol) were added. The solution was stirred at 60° C. for 4 hours and the the volatiles removed in vacuo. The residue was purified by column chromatography on silica gel using as eluant an ethyl acetate/hexanes gradient. This provided the desired product as a colourless oil, (0.938 g, 70%).

(c) (R/S)-3-Phenyl-5-piperazin-1-ylmethyl-oxazolidin-2-one

(R/S)-4-(2-Oxo-3-phenyl-oxazolidin-5-ylmethyl)-piperazine-1-carboxylic acid tert-butyl ester (0.93 g, 2.25 mmol) was hydrogenated in ethanol (50 mL) containing 10% Pd—C catalyst (0.1 g). The reaction was conducted at room temperature for 12 hours after which time the catalyst was removed by filtration through a pad of Celite®. The filtrate was concentrated in vacuo and the residue used without further purification.

(d) Title Compound

(R)-2-(6-Methoxyquinolin-4-yl)oxirane (Example 1b) (140 mg, 0.699 mmol) was dissolved in acetonitrile (10 mL) and (R/S)-3-Phenyl-5-piperazin-1-ylmethyl-oxazolidin-2-one (195 mg, 0.699 mmol) was added followed by lithium perchlorate (74 mg, 0.699 mmol). The slurry was stirred at room temperature for 12 hours after which time the volatiles were removed and the residue partitioned between brine and ethyl acetate. The organic phase was dried over magnesium sulfate and then concentrated in vacuo. The residue was purified on silica gel using a methanol/dichloromethane eluant. This provided a mixture of the title diastereomeric compounds as a colourless oil (47 mg, 14%).

δH (CD₃OD, 250 MHz) 8.80-8.72 (1H, m), 8.06-8.02 (1H, m), 7.63-7.60 (1H, m), 7.45-7.17 (5H, m), 6.88-6.82 (1H, m), 5.61-5.56 and 5.48-5.46 (1H, m), 4.874.70 (1H, m), 3.96 and 3.95 (3H, s), 4.15-3.65 and 2.90-2.45 (14H, m).

MS (APCI+) m/z 481 (MH+).

To a solution of the oil (47 mg) in dichloromethane (1 mL) was added methane sulfonic acid (1.96 mL of a 0.1M solution) in diethyl ether (10 mL) to generate the dimesylate salt. The title compound was isolated by centrifuigation, washing with diethyl ether and subsequent drying in vacuo.

### Example 17

### (S)-1-{4-[(R)-2-Hydroxy-2-(6-methoxy-quinolin-4-yl)-ethyl]-piperazin-1-yl}-3-phenylamino-propan-2-ol Dioxalate

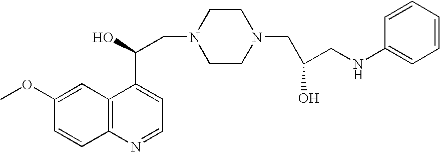

(a) (R)-1-(6-Methoxy-quinolin-4-yl)-2-(4-(R)-1-oxiranylmethyl-piperazin-1-yl)-ethanol

This compound was prepared in the same manner as (R)-1-(6-Methoxy-quinolin-4-yl)-2-(4-(S)-1-oxiranylmethyl-piperazin-1-yl)-ethanol (Example 1e) beginning from (S)-epichlorohydrin.

(b) Title Compound

(R)-1-(6-Methoxy-quinolin-4-yl)-2-(4-(R)-1-oxiranylmethyl-piperazin-1-yl)-ethanol (10 mg, 0.03 mmol) was dissolved in ethanol (0.5 mL) and aniline (20 mg, 0.22 mmol) was added. The solution was stirred at 60° C. for 12 hours after which time the volatiles were removed in vacuo and the residue purified by column chromatography on silica gel. Eluting with a gradient of methanoydichloromethane afforded the desired product (12 mg, 94%).

MS (APCI+) m/z 436 (MH+).

A solution of the oil (8 mg) in dichloromethane (1 mL) was added to oxalic acid (3.5 mg) in diethyl ether (10 mL) to generate the dioxalate salt. The title compound was isolated by centrifugation, washing with diethyl ether and subsequent drying in vacuo.

Biological Activity

The MIC (μg/ml) of test compounds against various organisms was determined: S. aureus Oxford, S. aureus WCUH29, S. pneumoniae 1629, S. pneumoniae N1387, S. pneumoniae ERY 2.

Examples 1-5, 7, 12 and 14-16 have an MIC of less than 1 μg/ml, and Examples 6, 8-11, 12 and 17 have an MIC of less than 81 g/ml against one or more of the above range of gram positive and gram negative bacteria.

1. A compound of formula (I) or a pharmaceutically acceptable derivative thereof: 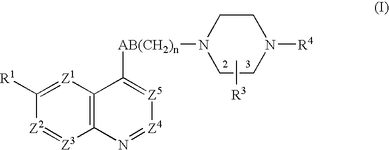
wherein:
one of Z¹, Z², Z³, Z⁴ and Z⁵ is N, one is CR^{1a} and the remainder are CH, or one of Z¹, Z², Z³, Z⁴ and Z⁵ is CR^{1a} and the remainder are CH;
R¹ and R^{1a} are independently selected from hydrogen; hydroxy; (C₁₋₆) alkoxy optionally substituted by (C₁₋₆)alkoxy, amino, piperidyl, guanidino or amidino any of which is optionally N-substituted by one or two (C₁₋₆)alkyl, acyl or (C₁₋₆)alylsulphonyl groups, (C₁₋₆)alkylthio, heterocyclylthio, heterocyclyloxy, arylthio, aryloxy, acylthio, acyloxy or (C₁₋₆)alkylsulphonyloxy; (C₁₋₆)alkoxy-substituted (C₁₋₆)alkyl; halogen; (C₁₋₆)alkyl; (C₁₋₆)alkylthio; trifluromethyl; nitro; azido; acyl; acyloxy; acylthio; (C₁₋₆)alkylsulphonyl; (C₁₋₆)alkylsulphoxide; arylsulphonyl; arylsulphoxide or an amino, piperidyl, guanidino or amidino group optionally N-substituted by one or two (C₁₋₆)alkyl, acyl or (C₁₋₆)alkylsulphonyl groups,
provided that when none of Z¹, Z², Z³, Z⁴ and Z⁵ is N, then R¹ is not hydrogen;
R³ is hydrogen; or
R³ is in the 2- or 3-position and is:
carboxy; (C₁₋₆)alkoxycarbonyl; aminocarbonyl wherein the amino group is optionally substituted by hydroxy, (C₁₋₆)alkyl, hydroxy(C₁₋₆)alkyl, aminocarbonyl(C₁₋₆)alkyl, (C₂₋₆)alkenyl, (C₁₋₆)alkylsulphonyl, trifluoromethylsulphonyl, (C₂₋₆)alkenylsulphonyl, (C₁₋₆)alkoxycarbonyl, (C₁₋₆)alkylcarbonyl, (C₂₋₆)alkenyloxycarbonyl or (C₂₋₆)alkenylcarbonyl and optionally further substituted by (C₁₋₆)alkyl, hydroxy(C₁₋₆)alkyl, aminocarbonyl(C₁₋₆)alkyl or (C₂₋₆)alkenyl; cyano; tetrazolyl; 2-oxo-oxazolidinyl optionally substituted by R¹⁰; 3-hydroxy-3-cyclobutene-1,2-dione-4-yl; 2,4-thiazolidinedione-5-yl; tetrazol-5-ylaminocarbonyl; 1,2,4-triazol-5-yl optionally substituted by R¹⁰; or 5-oxo-1,2,4-oxadiazol-3-yl; or
(C₁₋₄)alkyl or ethenyl optionally substituted with any of the groups listed above for R³ and/or 0 to 2 groups R¹² independently selected from:
halogen; (C₁₋₆)alkylthio; trifluoromethyl; (C₁₋₆)alkoxycarbonyl; (C₁₋₆)alkylcarbonyl; (C₂₋₆)alkenyloxycarbonyl; (C₂₋₆)alkenylcarbonyl; hydroxy optionally substituted by (C₁₋₆)alkyl, (C₂₋₆)alkenyl, (C₁₋₆)alkoxycarbonyl, (C₁₋₆)alkylcarbonyl, (C₂₋₆)alkenyloxycarbonyl, (C₂₋₆)alkenylcarbonyl or aminocarbonyl wherein the amino group is optionally substituted by (C₁₋₆)alkyl, (C₂₋₆)alkenyl, (C₁₋₆)alkylcarbonyl or (C₂₋₆)alkenylcarbonyl; amino optionally mono- or disubstituted by (C₁₋₆)alkoxycarbonyl, (C₁₋₆)alkylcarbonyl, (C₂₋₆)alkenyloxycarbonyl, (C₂₋₆)alkenylcarbonyl, (C₁₋₆)alkyl, (C₂₋₆)alkenyl, (C₁₋₆)alkylsulphonyl, (C₂₋₆)alkenylsulphonyl or aminocarbonyl wherein the amino group is optionally substituted by (C₁₋₆)alkyl or (C₂₋₆)alkenyl; aminocarbonyl wherein the amino group is optionally substituted by (C₁₋₆)alkyl, hydroxy(C₁₋₆)alkyl, aminocarbonyl(C₁₋₆)alkyl, (C₂₋₆)alkenyl, (C₁₋₆)alkoxycarbonyl, (C₁₋₆)alkylcarbonyl, (C₂₋₆)alkenyloxycarbonyl or (C₂₋₆)alkenylcarbonyl and optionally further substituted by (C₁₋₆)alkyl, hydroxy(C₁₋₆)alkyl, aminocarbonyl(C₁₋₆)alkyl or (C₂₋₆)alkenyl; oxo; (C₁₋₆)alkylsulphonyl; (C₂₋₆)alkenylsulphonyl; or (C₁₋₆)aminosulphonyl wherein the amino group is optionally substituted by (C₁₋₆)alkyl or (C₂₋₆)alkenyl;
in addition when R³ is disubstituted with a hydroxy or amino containing substituent and a carboxy containing substituent these may optionally together form a cyclic ester or amide linkage, respectively;
R¹⁰ is selected from (C₁₋₄)alkyl; (C₂₋₄)alkenyl and aryl any of which may be optionally substituted by a group R¹² as defined above; carboxy; aminocarbonyl wherein the amino group is optionally substituted by hydroxy, (C₁₋₆)alkyl, (C₂₋₆)alkenyl, (C₁₋₆)alkylsulphonyl, trifluoromethylsulphonyl, (C₂₋₆)alkenylsulphonyl, (C₁₋₆)alkoxycarbonyl, (C₁₋₆)alkylcarbonyl, (C₂₋₆)alkenyloxycarbonyl or (C₂₋₆)alkenylcarbonyl; and
R⁴ is a group —V—X¹—X²—X³—X⁴ in which:
V is CH₂, CO or SO₂;
X¹ is CR¹⁴R¹⁵;
X² is NR¹³, O, SO₂ or CR¹⁴R¹⁵;
X³ is NR¹³, O or CR¹⁴R¹⁵; wherein:
each of R¹⁴ and R¹⁵ is independently selected from: hydrogen; (C₁₋₄)alkoxy; (C₁₋₄)alkylthio; trifluoromethyl; cyano; (C₁₋₄)alkyl; (C₂₋₄)alkenyl; (C₁₋₄)alkoxycarbonyl; (C₁₋₄)alkylcarbonyl; (C₂₋₄)alkenyloxycarbonyl; (C₂₋₄)alkenylcarbonyl; hydroxy, amino or aminocarbonyl optionally substituted as for corresponding substituents in R³; (C₁₋₄)alkylsulphonyl; (C₂₋₄)alkenylsulphonyl; or aminosulphonyl wherein the amino group is optionally substituted by (C₁₋₄)alkyl or (C₂₋₄)alkenyl, provided that R¹⁴ and R¹⁵ on the same carbon atom are not both selected from optionally substituted hydroxy and optionally substituted amino; or
R¹⁴ and R¹⁵ together represent oxo;
R¹³ is hydrogen; trifluoromethyl, (C₁₋₆)alkyl; (C₂₋₆)alkenyl; (C₁₋₆)alkoxycarbonyl; (C₁₋₆)alkylcarbonyl; aminocarbonyl wherein the amino group is optionally substituted by (C₁₋₆)alkoxycarbonyl, (C₁₋₆)alkylcarbonyl, (C₂₋₆)alkenyloxycarbonyl, (C₂₋₆)alkenylcarbonyl, (C₁₋₆)alkyl or (C₂₋₆)alkenyl and optionally further substituted by (C₁₋₆)alkyl or (C₂₋₆)alkenyl;
two R¹⁴ groups or an R¹³ and an R¹⁴ group in X¹, X² and X³ together with the atoms to which they are attached and, if appropriate, the intervening group X² form a 5 or 6 membered carbocyclic or heterocyclic ring and the remaining R¹³, R¹⁴ and R¹⁵ groups are as above defined; or
two R¹⁴ groups or an R¹³ and an R¹⁴ group on adjacent atoms together represent a bond and the remaining R¹³, R¹⁴ and R¹⁵ groups are as above defined;
X⁴ is phenyl or C or N linked monocyclic aromatic 5- or 6-membered heterocycle containing up to four heteroatoms selected from O, S and N and optionally C-substituted by up to three groups selected from (C₁₋₄)alkylthio; halo; carboxy(C₁₋₄)alkyl; halo(C₁₋₄)alkoxy; halo(C₁₋₄)alkyl; (C₁₋₄)alkyl; (C₂₋₄)alkenyl; (C₁₋₄)alkoxycarbonyl; formyl; (C₁₋₄)alkylcarbonyl; (C₂₋₄)alkenyloxycarbonyl; (C₂₋₄)alkenylcarbonyl; (C₁₋₄)alkylcarbonyloxy; (C₁₋₄)alkoxycarbonyl(C₁₋₄)alkyl; hydroxy; hydroxy(C₁₋₄)alkyl; mercapto(C₁₋₄)alkyl; (C₁₋₄)alkoxy; nitro; cyano; carboxy; amino or aminocarbonyl optionally substituted as for corresponding substituents in R³; (C₁₋₄)alkylsulphonyl; (C₂₋₄)alkenylsulphonyl; or aminosulphonyl wherein the amino group is optionally substituted by (C₁₋₄)alkyl or (C₂₋₄)alkenyl; aryl, aryl(C₁₋₄)alkyl or aryl(C₁₋₄)alkoxy; and
optionally N substituted by trifluoromethyl; (C₁₋₄)alkyl optionally substituted by hydroxy, (C₁₋₆)alkoxy, (C₁₋₆)alkylthio, halo or trifluoromethyl; (C₂₋₄)alkenyl; aryl; aryl(C₁₋₄)alkyl; (C₁₋₄)alkoxycarbonyl; (C₁₋₄)alkylcarbonyl; formyl; (C₁₋₆)alkylsulphonyl; or aminocarbonyl wherein the amino group is optionally substituted by (C₁₋₄)alkoxycarbonyl, (C₁₋₄)alkylcarbonyl, (C₂₋₄)alkenyloxycarbonyl, (C₂₋₄)alkenylcarbonyl, (C₁₋₄)alkyl or (C₂₋₄)alkenyl and optionally further substituted by (C₁₋₄)alkyl or (C₂₋₄)alkenyl;
n is 0 and AB is NR¹¹CO, CO—CR⁸R⁹, CR⁶R⁷—CO, NR¹¹SO₂, CR⁶R⁷—SO₂ or CR⁶R⁷—CR⁸R⁹, provided that R⁸ and R⁹ are not optionally substituted hydroxy or amino and R⁶ and R⁸ do not represent a bond:
or n is 1 and AB is NR¹¹CO, CO—CR⁸R⁹, CR⁶R⁷—CO, NR¹¹SO₂, CONR¹¹, CR⁶R⁷—CR⁸R⁹, O—CR⁸R⁹ or NR¹¹—CR⁸R⁹;
and wherein:
each of R⁶ and R⁷, R⁸ and R⁹ is independently selected from: H; (C₁₋₆)alkoxy; (C₁₋₆)alkylthio; halo; trifluoromethyl; azido; (C₁₋₆)alkyl; (C₂₋₆)alkenyl; (C₁₋₆)alkoxycarbonyl; (C₁₋₆)alkylcarbonyl; (C₂₋₆)alkenyloxycarbonyl; (C₂₋₆)alkenylcarbonyl; hydroxy, amino or aminocarbonyl optionally substituted as for corresponding substituents in R³; (C₁₋₆)alkylsulphonyl; (C₂₋₆)alkenylsulphonyl; or (C₁₋₆)aminosulphonyl wherein the amino group is optionally substituted by (C₆)alkyl or (C₂₋₆)alkenyl;
or R⁶ and R⁸ together represent a bond and R⁷ and R⁹ are as above defined;
and each R¹¹ is independently H, trifluoromethyl, (C₁₋₆)alkyl, (C₂₋₆)alkenyl, (C₁₋₆)alkoxycarbonyl, (C₁₋₆)alkylcarbonyl, aminocarbonyl wherein the amino group is optionally substituted by (C₁₋₆)alkoxycarbonyl, (C₁₋₆)alkylcarbonyl, (C₂₋₆)alkenyloxycarbonyl, (C₂₋₆)alkenylcarbonyl, (C₁₋₆)alkyl or (C₂₋₆)alkenyl and optionally further substituted by (C₁₋₆)alkyl or (C₂₋₆)alkenyl;
or where one of R³ and R⁶, R⁷, R⁸ or R⁹ contains a carboxy group and the other contains a hydroxy or amino group they may together form a cyclic ester or amide linkage;
and each R¹¹ is independently H; trifluoromethyl; (C₁₋₆)alkyl; (C₂₋₆)alkenyl; (C₁₋₆)alkoxycarbonyl; (C₁₋₆)alkylcarbonyl; or aminocarbonyl wherein the amino group is optionally substituted by (C₁₋₆)alkoxycarbonyl, (C₁₋₆)alkylcarbonyl, (C₂₋₆)alkenyloxycarbonyl, (C₂₋₆)alkenylcarbonyl, (C₁₋₆)alkyl or (C₂₋₆)alkenyl and optionally further substituted by (C₁₋₆)alkyl or (C₂₋₆)alkenyl;
or where one of R³ and R⁶, R⁷, R⁸ or R⁹ contains a carboxy group and the other contains a hydroxy or amino group they may together form a cyclic ester or amide linkage.

2. A compound according to claim 1 wherein Z⁵ is CH or N, Z³ is CH or CF and Z¹, Z² and Z⁴ are each CH, or Z¹ is N, Z³ is CH or CF and Z², Z⁴ and Z⁵ are each CH.

3. A compound according to any preceding claim wherein R¹ is methoxy and R^{1a} is H or when Z³ is CR^{1a} it may be C—F.

4. A compound according to any preceding claim wherein R³ is hydrogen; CONH₂; 1-hydroxyalkyl; CH₂CO₂H; CH₂CONH₂; —CONHCH₂CONH₂; 1,2-dihydroxyalkyl; CH₂CN; 2-oxo-oxazolidin-5-yl or 2-oxo-oxazolidin-5-yl(C₁₋₄alkyl).

5. A compound according to any preceding claim wherein n is 0 and either A is CHOH and B is CH₂ or A is NH and B is CO.

6. A compound according to any preceding claim wherein V is CH₂ and —X¹—X²—X³—is CH₂)₂—O—, —CH₂—CH═CH—, —(CH₂)₃—, CH₂)₂—NH—, —CH(OH)—CH₂—NH—, —CH₂NHCO— or —CH₂CONH—.

7. A compound according to any preceding claim wherein X⁴ is 2-pyridyl, 3-fluorophenyl, 3,5-difluorophenyl or 1,3-thiazole-2-yl.

8. A compound according to claim 1 selected from:
(R)-1-(3,5-Difluoro-phenylamino)-3-{4-[(R)-2-hydroxy-2-(6-methoxy-quinolin-4-yl)-ethyl]-piperazin-1-yl}-propan-2-ol
(R/S)-1-(3,5-Difluoro-phenylamino)-3-{4-[(R)-2-hydroxy-2-(6-methoxy-quinolin-4-yl)-ethyl]-piperazin-1-yl}-propan-2-ol
3-(3,5-Difluoro-phenyl)-5-{4-[(R)-2-hydroxy-2-(6-methoxy-quinolin-4-yl)-ethyl]-piperazin-1-ylmethyl}-oxazolidin-2-one
N-(2-{4-[(R)-2-Hydroxy-2-(6-methoxy-quinolin-4-yl)-ethyl]-piperazin-1-yl}-ethyl)-benzamide
3,5-Difluoro-N-(2-{4-[(R)-2-hydroxy-2-(6-methoxy-quinolin-4-yl)-ethyl]-piperazin-1-yl}-ethyl)-benzamide
Pyridine-2-carboxylic acid (2-{4-[(R)-2-hydroxy-2-(6-methoxy-quinolin-4-yl)-ethyl]-piperazin-1-yl}-ethyl)-amide
Thiophene-2-carboxylic acid (2-{4-[(R)-2-hydroxy-2-(6-methoxy-quinolin-4-yl)-ethyl]-piperazin-1-yl}-ethyl)-amide
Furan-2-carboxylic acid (2-{4-[(R)-2-hydroxy-2-(6-methoxy-quinolin-4-yl)-ethyl]-piperazin-1-yl}-ethyl)-amide
N-(2-{4-[( )-2-Hydroxy-2-(6-methoxy-quinolin-4-yl)-ethyl]-piperazin-1-yl}-ethyl)-trifluoromethyl-benzamide
(E)-1-{4-[(R)-2-Hydroxy-2-(6-methoxy-quinolin-4-yl)-ethyl]-piperazin-1-yl}-4-phenyl-but-3-en-1-one
(R)-1-(6-Methoxy-quinolin-4-yl)-2-[4-(4-phenyl-butyl)-piperazin-1-yl]-ethanol
(S)-1-(6-Methoxy-quinolin-4-yl)-2-[4-(4-phenyl-butyl)-piperazin-1-yl]-ethylamine
N-(3,5-Difluoro-phenyl)-3-{4-[(R)-2-hydroxy-2-(6-methoxy-quinolin-4-yl)-ethyl]-piperazin-1-yl }-propionamide
1-{4-[(R)-2-Hydroxy-2-(6-methoxy-quinolinyl)-ethyl]-piperazin-1-yl}-3-phenoxy-propan-2-ol
3-(3-Fluoro-phenyl)-5-{4-[(R)-2-hydroxy-2-(6-methoxy-quinolin-4-yl)-ethyl]-piperazin-1-ylmethyl}-oxazolidin-2-one and
(S)-1-{4-[(R)-2-Hydroxy-2-(6-methoxy-quinolin-4-yl)ethyl]-piperazin-1-yl}-3-phenylamino-propan-2-ol
or a pharmaceutically acceptable derivative thereof.

9. A method of treatment of bacterial infections in mammals, particularly in man, which method comprises the administration to a mammal in need of such treatment an effective amount of a compound according to claim 1.

10. The use of a compound according to claim 1, in the manufacture of a medicament for use in the treatment of bacterial infections in mammals.

11. A pharmaceutical composition comprising a compound according to claim 1, and a pharmaceutically acceptable carrier.

12. A process for preparing a compound according to claim 1, which process comprises reacting a compound of formula (IV) with a compound of formula (V): 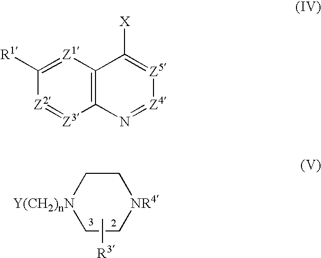
wherein n is as defined in formula (I); Z^{1′}, Z^{2′}, Z^{3′}, Z^{4′}, Z^{5′}, R^{1′}, R^{3′} and R^{4′} are Z¹, Z², Z³, Z⁴, Z⁵, R¹, R³ and R⁴ as defined in formula (I) or groups convertible thereto;
and X and Y may be the following combinations:
wherein n is as defined in formula (I); Z^{1′}, Z^{2′}, Z^{3′}, Z^{4′}, Z^{5′}, R^{1′}, R^{3′} and R^{4′} are Z¹, Z², Z³, Z⁴, Z⁵, R¹, R³ and R⁴ as defined in formula (I) or groups convertible thereto;
and X and Y may be the following combinations:
(i) X is A′—COW, Y is H and n is 0;
(ii) X is CR⁶═CR⁸R⁹, Y is H and n is 0;
(iii) X is oxirane, Y is H and n is 0;
(iv) X is N═C═O and Y is H and n is 0;
(v) one of X and Y is CO₂R^{y} and the other is CH₂CO₂R^{x};
(vi) X is CHR⁶R⁷ and Y is C(═O)R⁸;
(vii) X is CR⁷—PR^{z}₃ and Y is C(═O)R⁹ and n=1;
(viii) X is C(═O)R⁷ and Y is CR⁹═PR^{z}₃ and n=1;
(ix) Y is COW and X is NHR^{11′}NCO or NR^{11′}COW and n=0 or 1 or when n=1 X is COW and Y is NHR^{11′}, NCO or NR^{11′}COW;
(x) X is NHR^{11′} and Y is C(═O)R⁸ and n=1;
(xi) X is NHR^{11′} and Y is CR⁸R⁹W and n=1;
(xii) X is NR^{11′}COCH₂W or NR^{11′}SO₂CH₂W and Y is H and n=0;
(xiii) X is CR⁶R⁷SO₂W and Y is H and n=0;
(xiv) X is W or OH and Y is CH₂OH and n is 1;
(xv) X is NHR^{11′} and Y is SO₂W or X is NR^{11′}SO₂W and Y is H, and n is 0;
in which W is a leaving group, e.g. halo or imidazolyl; R^{x} and R^{y} are (C₁₋₆)alkyl; R^{z} is aryl or (C₁₋₆)allyl; A′ and NR^{11′} are A and NR¹¹ as defined in formula (I), or groups convertible thereto; and oxirane is: 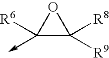
wherein R⁶, R⁸ and R⁹ are as defined in formula (I);
and thereafter optionally or as necessary converting A′, Z^{1′}, Z^{2′}, Z^{3′}, Z^{4′}, Z^{5′}, R^{1′}, R^{3′}, R^{4′} and NR^{11′}; to A, Z¹, Z², Z³, Z⁴, Z⁵, R¹, R³, R⁴ and NR^{11′}; converting A-B to other A-B, interconverting R¹, R³ and/or R⁴, and/or forming a pharmaceutically acceptable derivative thereof.
